(19) United States
(12) Patent Application Publication
(10) Pub. No.: US 2026/0016439 A1
(43) Pub. Date: Jan. 15, 2026

(54) CNT FILM USING CLICK REACTION, CNT-BASED BIOSENSOR USING SAME, AND MANUFACTURING METHOD THEREFOR

(71) Applicant: KOREA RESEARCH INSTITUTE OF CHEMICAL TECHNOLOGY, Daejeon (KR)

(72) Inventors: Bogyu LIM, Daejeon (KR); Jong Mok PARK, Daejeon (KR); Seo Hyun JUNG, Daejeon (KR); Yu Jin JUNG, Daejeon (KR); Ye Jin KIM, Daejeon (KR); Henok GIRMA, Daejeon (KR); Ga Young KIM, Daejeon (KR); Kwang Hun PARK, Daejeon (KR)

(73) Assignee: KOREA RESEARCH INSTITUTE OF CHEMICAL TECHNOLOGY, Daejeon (KR)

(21) Appl. No.: 19/155,319

(22) PCT Filed: Mar. 14, 2023
(86) PCT No.: PCT/KR2023/003409

§ 371 Date: Aug. 11, 2025

(30) Foreign Application Priority Data
Mar. 18, 2022 (KR) 10-2022-0033915

(51) Int. Cl. G01N 27/327; C08L 33/14; G01N 33/543

(52) U.S. Cl. CPC G01N 27/327; C08L 33/14; G01N 33/5438

(57) ABSTRACT

A CNT biosensor according to the present invention has a CNT film that is uniformly formed with high density by using a click reaction and thus can have high stability against water or an organic solvent. In particular, CNT films and biosensors that are conventionally manufactured by spray coating and spin coating of a CNT solution cannot secure reproducibility and reliability due to large differences in properties between devices, but the present invention can provide a CNT biosensor having high reproducibility and reliability through a comparatively simple method.

### TECHNICAL FIELD

The present invention relates to a CNT film using a click reaction and a CNT-based biosensor using the same, and more particularly, to a CNT-based biosensor manufactured using a CNT film having excellent stability against water or an organic solvent and easily allowing density control through a click reaction, and a method for manufacturing the same.

### BACKGROUND ART

A biosensor is a semiconductor device designed to detect biological reactions and use a transducer to convert the biological reactions into electrical signals. The first semiconductor device using carbon nanotubes (CNTs) was developed in 1997, and studies on next-generation nano-semiconductor devices that may solve size issues caused by the high integration and high functionality required for semiconductor devices using silicon substrates have been actively conducted.

Accordingly, in order to manufacture a CNT semiconductor device, there are a method of immersing the entire substrate in a CNT solution and spin-coating the CNT solution onto a substrate layer, a method of directly patterning a carbon nanotube material on a substrate using a printing process, and the like. However, all of the above methods have a problem in that the bonding strength between the substrate and the CNT is insufficient, and thus the CNT is easily peeled off by water or an organic solvent during a washing process. This problem has a fatal disadvantage in that solvent stability is reduced, leading to issues such as CNT film peeling during the washing process, which is an essential step in the process of manufacturing a biosensor. In addition, a method for manufacturing a CNT film that is generally formed by a random network has a problem in that the reliability of the manufactured devices is reduced due to non-uniform connectivity between CNTs within the film, making it difficult to easily secure commercialization.

Therefore, there is an urgent need for research and development of a CNT-based biosensor that has excellent adhesion to a substrate, has excellent reliability between devices due to uniformity of CNT films formed at a high density, and has excellent stability against water or an organic solvent while maintaining the electrical characteristics of the CNT.

### DISCLOSURE

### Technical Problem

In order to solve the problems of the related art, an object of the present invention is to provide a CNT-based biosensor having excellent stability against water or an organic solvent and high reliability by growing CNTs fixed to a substrate using a click reaction and implementing a high CNT density between films by controlling the CNT density through controlling a click reaction degree.

In addition, another object of the present invention is to provide a method for manufacturing a semiconductor device and a CNT-based biosensor that are relatively easy to process using a click reaction.

In addition, still another object of the present invention is to provide a method for detecting a target biomarker using the CNT-based biosensor described above.

### Technical Solution

In order to achieve the object, as a result of continuously conducting studies to develop a CNT biosensor having excellent stability against water or an organic solvent and high reliability between devices due to excellent adhesion between a CNT film and a substrate and a method for manufacturing the same, the present inventors surprisingly have found that when a CNT biosensor is manufactured using a click reaction, it is possible to manufacture a CNT biosensor in which a CNT film is not peeled off even after washing due to the CNT film formed uniformly at a high density and excellent stability against water or an organic solvent and has excellent reliability between devices, thereby completing the present invention.

In one general aspect, a CNT biosensor includes a polymer layer formed of a first polymer on a substrate; a composite layer formed of a second polymer-CNT composite on the polymer layer; a metal electrode formed on the composite layer; and an antibody layer formed on the composite layer, wherein the second polymer-CNT composite is a composite in which a CNT is wrapped by a second polymer, and the polymer layer and the composite layer are connected via triazole.

In one embodiment of the present invention, the composite layer and the antibody layer may be connected by a linker.

In one embodiment of the present invention, the linker may be connected to the composite layer by a covalent bond or a non-covalent bond.

In one embodiment of the present invention, the triazole may be represented by the following Chemical Formula 1:

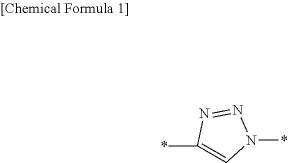 [SMILES: *c1cn(*)nn1]

wherein
the asterisks (*) are each independently a connection point with the first polymer of the polymer layer or the second polymer of the composite layer, and the two asterisks (*) are connection points between different layers.

In one embodiment of the present invention, the first polymer may be an acrylic-based copolymer.

In one embodiment of the present invention, the first polymer may be represented by the following Chemical Formula 2, the second polymer may be represented by the following Chemical Formula 3, the triazole may be formed by a click reaction between the first polymer and the second polymer, and the click reaction may be a reaction represented by the following Reaction Formula 1:

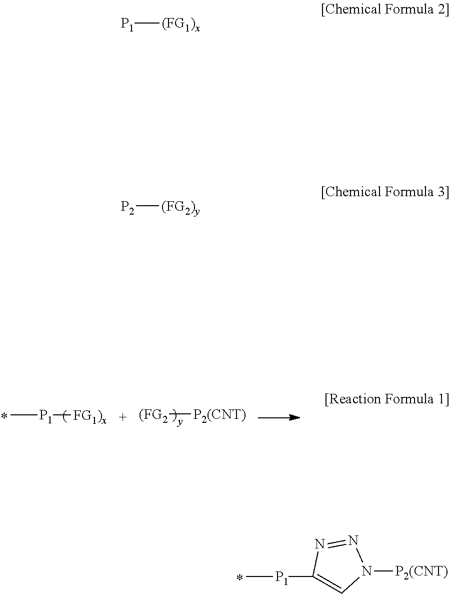

wherein
P₁ is a residue derived from the first polymer;
P₂ is a residue derived from the second polymer;
* is a moiety where P₁ is fixed to the substrate;
P₂(CNT) is a residue derived from the second polymer-CNT composite;
FG₁ is an alkynyl functional group;
FG₂ is an azide functional group; and
x and y are integers of 1 or more.

In one embodiment, Chemical Formula 2 may be represented by the following Chemical Formula 4 or Chemical Formula 5:

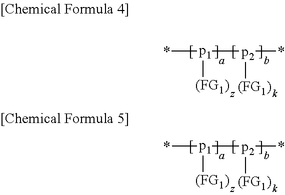

wherein
FG₁ is an alkynyl functional group;
FG₃ is an epoxy functional group;
p₁ and p₂ are repeating units derived from a monomer having an FG₁ functional group at the end;
p₃ is a repeating unit derived from a monomer having an FG₃ functional group at the end;
z, k, and t are integers of 1 to 7; and
a, b, and c are integers of 1 or more.

In one embodiment of the present invention, Chemical Formula 4 may be represented by the following Chemical Formula 6, and Chemical Formula 5 may be represented by the following Chemical Formula 8:

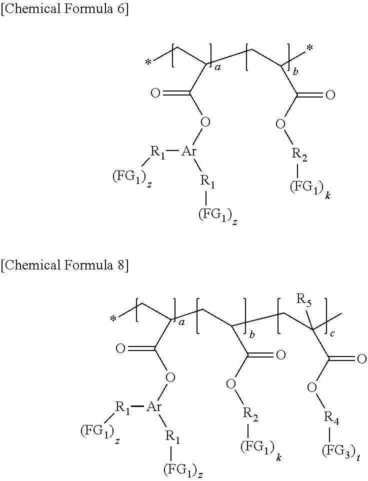

wherein
Ar is a trivalent aromatic radical;
R₁, R₂, and R₄ are independently C₁₋₅₀ alkylene, C₃₋₅₀ cycloalkylene, C₆₋₅₀ arylene, C₃₋₅₀ heteroarylene, C₁₋₅₀ alkoxycarbonylene, or a combination thereof;
the alkylene, cycloalkylene, arylene, heteroarylene, and alkoxycarbonylene may be optionally substituted with one or more selected from hydroxy, halogen, nitro, cyano, amino, carboxyl, carboxylate, C₁₋₂₀ alkyl, C₂₋₂₀ alkenyl, C₂₋₂₀ alkynyl, C₁₋₂₀ haloalkyl, C₁₋₂₀ alkoxy, C₁₋₂₀ alkoxycarbonyl, C₃₋₃₀ cycloalkyl, (C₆₋₃₀)ar(C₁₋₂₀)alkyl, C₆₋₃₀ aryl, and C₃₋₃₀ heteroaryl;
FG₁ is an alkynyl functional group;
FG₃ is an epoxy functional group;
z, k, and t are independently an integer of 1 to 7; and
a, b, and c are independently an integer of 1 or more.

In one embodiment of the present invention, Chemical Formula 6 may be represented by the following Chemical Formula 7, and Chemical Formula 8 may be represented by the following Chemical Formula 9:

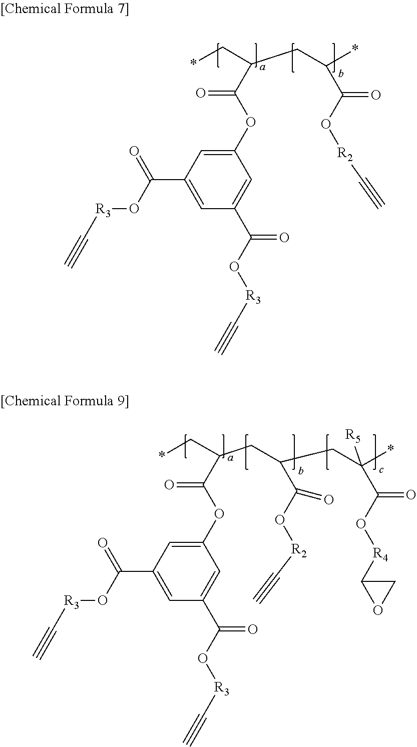

wherein
R₂ to R₄ are independently C₁₋₁₀ alkylene;
R₅ is hydrogen or methyl; and
a, b, and c are independently an integer of 1 or more.

In one embodiment of the present invention, in the CNT biosensor, the second polymer may be a fluorene-based copolymer.

In one embodiment of the present invention, Chemical Formula 3 may be a copolymer containing a repeating unit (n) of the following Chemical Formula 10 and a repeating unit (m) of the following Chemical Formula 11:

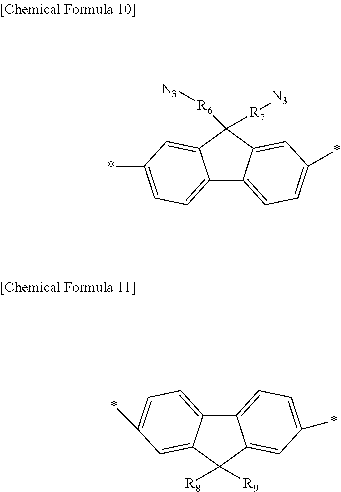

wherein
R₆ and R₇ are independently C₅₋₅₀ alkylene; and
R₈ and R₉ are independently C₅₋₅₀ alkyl.

In one embodiment of the present invention, the CNT in the second polymer-CNT composite may be a semiconducting single-walled carbon nanotube (sc-SWCNT).

In one embodiment of the present invention, the antibody layer may include an antibody that specifically binds to a target biomarker.

In one embodiment of the present invention, the target biomarker may be one or a combination of two or more selected from a biomarker for predicting metabolic syndrome, a biomarker for predicting severe liver fibrosis, a biomarker for diagnosing cardiovascular disease, a biomarker for diagnosing cancer, a biomarker for diagnosing obesity, and a biomarker for predicting or diagnosing neurodegenerative disease.

In one embodiment of the present invention, the CNT biosensor may be used for detecting a target biomarker.

In another general aspect, there is provided a method for manufacturing a CNT biosensor, the CNT biosensor including a polymer layer formed of a first polymer on a substrate; a composite layer formed of a second polymer-CNT composite on the polymer layer; a metal electrode formed on the composite layer; and an antibody layer formed on the composite layer,
wherein the second polymer-CNT composite is a composite in which a CNT is wrapped by a second polymer, and the polymer layer and the composite layer are connected via triazole.

In one embodiment of the present invention, the method for manufacturing a CNT biosensor may include:
(a) coating and fixing a first polymer on a substrate;
(b) immersing the substrate coated with the first polymer in a second polymer-CNT composite solution;
(c) forming a polymer layer and a composite layer by a click reaction between the first polymer and the second polymer;
(d) forming a source electrode and a drain electrode on the composite layer; and
(e) forming an antibody layer on the composite layer.

In one embodiment of the present invention, the step (e) may include (e-1) introducing a linker onto the composite layer; and (e-2) forming an antibody layer by reacting the linker and an antibody.

In one embodiment of the present invention, the linker may be represented by the following Chemical Formula 15:

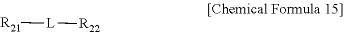

wherein
R₂₁ is

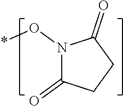

or a polycyclic aromatic hydrocarbon,
L is

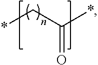

R₂₂ is

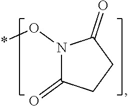

and
n is an integer of 1 or more.

In one embodiment of the present invention, in the step (e-1), R₂₁ of the linker may be covalently bonded through a chemical reaction with the second polymer of the composite layer or non-covalently bonded through a n-n interaction with the CNT of the composite layer.

In one embodiment of the present invention, in the step (e-1), R₂₂ of the linker may be covalently bonded through a reaction with the antibody.

In one embodiment of the present invention, the step (a) may include:
(a-1) washing the substrate with a solvent;
(a-2) coating a self-assembled monolayer (SAM);
(a-3) coating the first polymer;
(a-4) performing UV curing; and
(a-5) washing a compound unfixed to the substrate with a solvent.

In one embodiment of the present invention, in the step (a-3), the first polymer may be represented by the following Chemical Formula 4:

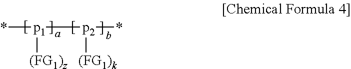

wherein
FG₁ is an alkynyl functional group;
p₁ and p₂ are repeating units derived from a monomer having an FG₁ functional group at the end;
z and k are integers of 1 to 7; and
a and b are integers of 1 or more.

In one embodiment of the present invention, the step (a-4) may further include forming a pattern.

In one embodiment of the present invention, the step (a) may include:
(a′-1) washing the substrate with a solvent;
(a′-2) coating the first polymer;
(a′-3) performing a heat treatment; and
(a′-4) washing a compound unfixed to the substrate with a solvent.

In one embodiment of the present invention, in the step (a′-2), the first polymer may be represented by the following Chemical Formula 5:

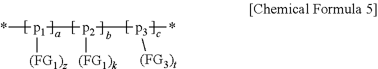

wherein
FG₁ is an alkynyl functional group;
FG₃ is an epoxy functional group;
p₁ and p₂ are repeating units derived from a monomer having an FG₁ functional group at the end;
p₃ is a repeating unit derived from a monomer having an FG₃ functional group at the end;
z, k, and t are integers of 1 to 7; and
a, b, and c are integers of 1 or more.

### Advantages of the Invention

The CNT biosensor according to the present invention includes a CNT film that is uniformly formed at a high density by using a click reaction and thus may have high stability against water or an organic solvent. In particular, while a conventional CNT biosensor manufactured by spray coating and spin coating a CNT solution had large differences in physical properties between devices and thus could not secure reproducibility and reliability, the CNT biosensor according to the present invention has the advantage of being able to manufacture a CNT biosensor having high reproducibility and high reliability by a relatively simple method, thereby easily securing commercialization.

In addition, in the case of the method for manufacturing a CNT biosensor according to the present invention, a CNT film having a desired density may be manufactured by controlling the time of the click reaction, and a biosensor coated with CNTs at a high density may be obtained in a short reaction time, such that there is an advantage of a simple manufacturing process.

Accordingly, the CNT biosensor according to the present invention may effectively implement excellent adhesion to a substrate, a high density, uniformity, high stability against water or an organic solvent, high reproducibility, process easiness, and reliability.

### DESCRIPTION OF DRAWINGS

FIG. 1 is a schematic diagram showing a method for manufacturing a CNT biosensor and a biomarker detection experiment process according to one embodiment of the present invention.

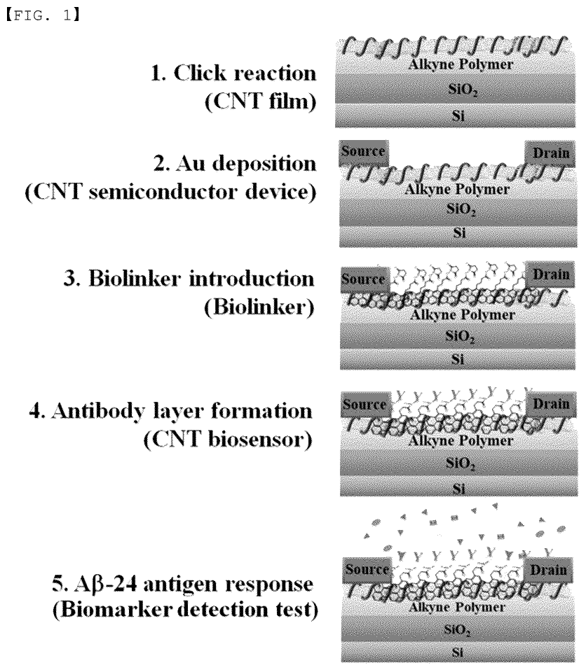

FIG. 2 is an image obtained by measuring a contact angle of a coating layer after coating of a self-assembled monolayer (SAM) in Example 1 of the present invention.

FIG. 3A is a graph showing ultraviolet-visible spectroscopy (UV-Vis spectroscopy) results of an acrylate copolymer (i) solution coated in Example 1 according to the present invention before and after being washed with a solvent after UV curing, and FIG. 3B is a graph showing ultraviolet-visible spectroscopy analysis results of an acrylate copolymer (ii) solution coated in Example 2 according to the present invention before and after being washed with a solvent after heat curing.

FIG. 4 is a diagram showing a shadow mask used in Example 1 and a CNT semiconductor device according to Example 1.

FIG. 5 illustrates graphs showing electrical characteristic curves (output curves and transfer curves) for CNT semiconductor devices according to Examples 1 and 2 and Comparative Example 1.

FIG. 6 is an SEM image of a surface of the CNT semiconductor device measured after introducing a linker in Example 1 of the present invention.

FIG. 7 is a graph showing an average resistance value of samples for each process for manufacturing a CNT biosensor in Example 1 of the present invention, and the number of measured devices is 60.

### BEST MODE

Hereinafter, a CNT biosensor using a click reaction according to the present invention and a method for manufacturing the same will be described in detail. In this case, unless otherwise defined, all the technical terms and scientific terms used herein have the general meanings as commonly understood by those skilled in the art to which the present invention pertains, and the description for the known function and configuration unnecessarily obscuring the gist of the present invention will be omitted in the following description.

Unless the context clearly indicates otherwise, singular forms used in the present specification may be intended to include plural forms.

In addition, a unit used in the present specification without particular mention is based on a weight, and as an example, a unit of % or a ratio refers to wt % or a weight ratio and wt % refers to wt % of any one component in the entire composition, unless otherwise defined.

In addition, a numerical range used in the present specification includes upper and lower limits and all values within these limits, increments logically derived from a form and span of a defined range, all double limited values, and all possible combinations of the upper and lower limits in the numerical range defined in different forms. Unless otherwise specifically defined in the present specification of the present invention, values out of a numerical range that may occur due to experimental errors or rounded values also fall within the defined numerical range.

In addition, in the present invention, when a layer is referred to as being “on” another layer, it may include not only the case of being in contact with another layer but also the case in which one or more other layers are interposed between the two layers.

The term “polymer” in the present specification includes a polymer and a copolymer.

The term “copolymer” in the present specification generally means any polymer derived from more than one kind of monomer, in which the polymer contains more than one kind of corresponding repeating unit. The copolymer is a reaction product of two or more kinds of monomers and may thus contain two or more kinds of corresponding repeating units. The copolymer may be present as a block copolymer, a random copolymer, and/or an alternating copolymer.

The term “acrylic-based” in the present specification includes both methacrylic-based and acrylic-based.

The term “acrylate” in the present specification includes both methacrylate and acrylate.

The term “residue” in the present specification means a moiety remaining in a polymer excluding a specific functional group, and the type of the polymer is not particularly limited.

The term “wrapping” in the present specification means that a polymer wraps a CNT by an electrostatic interaction, and may include the meanings of coating, application, bonding, and attachment. In addition, the electrostatic interaction may refer to a n-electron interaction (n-n stacking interaction).

The term “alkyl” in the present specification includes both a linear chain form and a branched chain form, and may have 1 to 30 carbon atoms, and specifically, 1 to 20 carbon atoms.

The terms “halogen” and “halo” in the present specification refer to fluorine, chlorine, bromine, or iodine.

The term “haloalkyl” in the present specification refers to an alkyl group in which one or more hydrogen atoms are substituted with a halogen atom, respectively. For example, the haloalkyl includes —CF₃, —CHF₂, —CH₂F, —CBr₃, —CHBr₂, —CH₂Br, —CCl₃, —CHCl₂, —CH₂CI, —CI₃, —CHI₂, —CH₂I, —CH₂—CF₃, —CH₂—CHF₂, —CH₂—CH₂F, —CH₂—CBr₃, —CH₂—CHBr₂, —CH₂—CH₂Br, —CH₂—CCl₃, —CH₂—CHCl₂, —CH₂—CH₂CI, —CH₂—CI₃, —CH₂—CHI₂, —CH₂—CH₂I, and the like. Herein, alkyl and halogen are as defined above.

The term “alkenyl” in the present specification refers to a saturated linear chain or branched chain acyclic hydrocarbon having 2 to 30, and specifically, 2 to 20 carbon atoms, and at least one carbon-carbon double bond.

The term “alkynyl” in the present specification refers to a saturated linear chain or branched chain acyclic hydrocarbon having 2 to 30, and specifically, 2 to 20 carbon atoms, and at least one carbon-carbon triple bond.

The term “alkoxy” in the present specification refers to —O-(alkyl) including —OCH₃, —OCH₂CH₃, —O(CH₂)₂CH₃, —O(CH₂)₃CH₃, —O(CH₂)₄CH₃, —O(CH₂)₅CH₃, and the like, in which alkyl is as defined above.

The term “aryl” in the present specification refers to a carbocyclic aromatic group containing 5 to 10 ring atoms. A representative example includes phenyl, tolyl, xylyl, naphthyl, tetrahydronaphthyl, anthracenyl, fluorenyl, indenyl, azulenyl, and the like, but is not limited thereto. Furthermore, aryl includes a carbocyclic aromatic group being linked by alkylene or alkenylene or being linked by one or more heteroatoms selected from B, O, N, C(═O), P, P(═O), S, S(═O)₂, and a Si atom.

The term “alkoxycarbonyl” in the present specification refers to an alkoxy-C(═O)—* radical, in which “alkoxy” is as defined above. Examples of the alkoxycarbonyl radical include methoxycarbonyl, ethoxycarbonyl, isopropoxycarbonyl, propoxycarbonyl, butoxycarbonyl, isobutoxycarbonyl, and t-butoxycarbonyl, but are not limited thereto.

The term “cycloalkyl” in the present specification refers to a monocyclic or polycyclic saturated ring having carbon and hydrogen atoms and no carbon-carbon multiple bond. Examples of the cycloalkyl group include C₃₋₁₀ cycloalkyl (for example, cyclopropyl, cyclobutyl, cyclopentyl, cyclohexyl, and cycloheptyl), but are not limited thereto. The cycloalkyl group may be optionally substituted. In one embodiment, the cycloalkyl group is a monocyclic or bicyclic ring.

The term “aralkyl” in the present specification is alkyl in which one or more hydrogens are substituted with aryl, and includes benzyl and the like.

The terms “alkylene”, “alkenylene”, “alkynylene”, “cycloalkylene”, “arylene”, “heteroarylene”, and “alkoxycarbonylene” in the present specification refer to divalent organic radicals derived by removing one hydrogen from “alkyl”, “alkenyl”, “alkynyl”, “cycloalkyl”, “aryl”, “heteroaryl”, and “alkoxycarbonyl”, and follow the definitions of alkyl, alkenyl, alkynyl, cycloalkyl, aryl, heteroaryl, and alkoxycarbonyl.

In the present specification, the term “hydroxy” refers to —OH, the term “nitro” refers to —NO₂, the term “cyano” refers to —CN, the term “amino” refers to —NH₂, the term “carboxyl” refers to —COOH, and the term “carboxylic acid salt” refers to —COOM. M may be an alkali metal or an alkali earth metal.

The term “alkali metal” in the present specification refers to lithium (Li), sodium (Na), potassium (K), rubidium (Rb), cesium (Cs), and francium (Fr), which are chemical elements of Group 1 of the periodic table except hydrogen, and the term “alkali earth metal” in the present specification refers to beryllium (Be), magnesium (Mg), calcium (Ca), strontium (Sr), barium (Ba), and radium (Ra), which are Group 2 elements of the periodic table.

The term “comprise(s)” described in the present invention is an open-ended description having a meaning equivalent to the term such as “include(s)”, “contain(s)”, “have(has)”, or “is/are characterized”, and does not exclude elements, materials, or processes which are not further listed.

Hereinafter, a CNT biosensor according to one embodiment of the present invention will be described in detail.

The present invention provides a CNT biosensor including a polymer layer formed of a first polymer on a substrate; a composite layer formed of a second polymer-CNT composite on the polymer layer; a metal electrode formed on the composite layer; and an antibody layer formed on the composite layer. Specifically, the second polymer-CNT composite may be a composite in which a CNT is wrapped by a second polymer, and the polymer layer and the composite layer may be connected via triazole.

The substrate may be an inorganic substrate including glass, quartz, silicon, and the like, or an organic substrate including polyethylene terephthalate, polyethylene sulfone, polycarbonate, polystyrene, polypropylene, polyester, polyimide, polyetheretherketone, polyetherimide, an acryl resin, an olefin maleimide copolymer, and the like, but is not limited thereto. In addition, the substrate may be a usual silicon wafer, a substrate including an oxide film formed on the inorganic substrate, or a flexible substrate including the organic substrate, plastic, and the like, but is not particularly limited as long as a CNT film may be formed on the substrate. Additionally, in order to improve adhesion between the substrate and the CNT film, a physical or chemical treatment may be performed. The CNT film may be formed on the substrate and may be applied to a semiconductor device, a transparent electrode, a display, and the like.

In the CNT biosensor according to one embodiment of the present invention, the triazole may be represented by the following Chemical Formula 1.

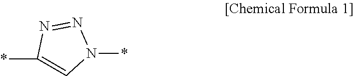 [SMILES: *c1cn(*)nn1]

In Chemical Formula 1, the asterisks (*) are each independently a connection point with the first polymer of the polymer layer or the second polymer of the composite layer, and the two asterisks (*) are connection points between different layers.

In the CNT biosensor according to one embodiment of the present invention, the first polymer may be a polymer containing an alkynyl functional group and may be represented by the following Chemical Formula 2, the second polymer may be a polymer containing an azide functional group and may be represented by the following Chemical Formula 3, the triazole may be formed by a click reaction between the first polymer and the second polymer, and the click reaction may be represented by the following Reaction Formula 1.

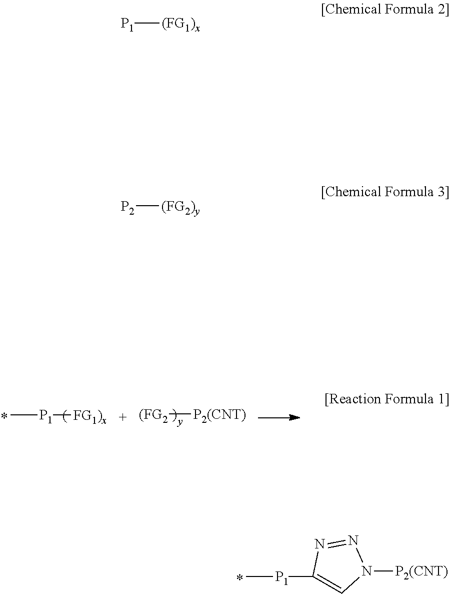

In Chemical Formulas 2 and 3 and Reaction Formula 1, P₁ is a residue derived from the first polymer, P₂ is a residue derived from the second polymer, * is a moiety where P₁ is fixed to the substrate, P₂(CNT) is a residue derived from the second polymer-CNT composite, FG₁ is an alkynyl functional group, FG₂ is an azide functional group, and x and y are integers of 1 or more.

The residue derived from the first polymer refers to a moiety remaining in the first polymer excluding the FG₁ functional group, and the first polymer is the same as described below.

The residue derived from the second polymer refers to a moiety remaining in the second polymer excluding the FG₂ functional group, and the second polymer is the same as described below.

The residue derived from the second polymer-CNT composite refers to a moiety remaining in the second polymer-CNT composite excluding the FG₂ functional group, and the second polymer-CNT composite is the same as described below.

Reaction Formula 1 may be specifically represented by the following Reaction Formula 2.

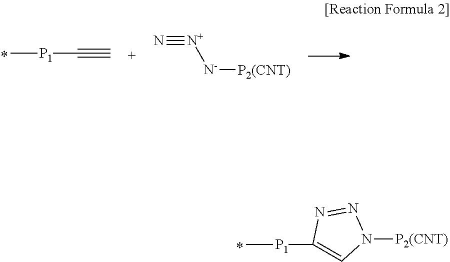

As seen in Reaction Formulas 1 and 2, the alkynyl functional group of Chemical Formula 2 and the azide functional group of Chemical Formula 3 may form a triazole ring by a click reaction in the presence of a copper catalyst. P₁ and P₂(CNT) are chemically bonded by the triazole ring, such that a polymer layer and a composite layer may be formed on the substrate. The type of first polymer is not particularly limited as long as it has an alkynyl functional group, and the type of second polymer is also not limited as long as it has an azide functional group.

Specifically, according to one embodiment of the present invention, the first polymer may be used without significant limitation in type as long as it has hydroxy, epoxy, carboxyl, thiol, alkene, and alkynyl functional groups, and specifically epoxy and alkynyl functional groups, in the side chain. Specifically, the first polymer may be an acrylic-based copolymer, the acrylic-based copolymer may be obtained by polymerizing two or more monomers, and the monomer may be an acrylic-based monomer or a methacrylic-based monomer. The monomer may have hydroxy, epoxy, carboxyl, thiol, alkene, and alkynyl as functional groups, and specifically may have epoxy and alkynyl functional groups, at the end. The monomer may be used by direct synthesis or may be a commercially available product, but is not limited thereto.

In addition, the acrylic-based copolymer may be synthesized by a commonly used polymerization method. Specifically, the polymerization method may be solution polymerization, but is not limited thereto. The solution polymerization may be polymerization performed by including the monomer, an initiator, and a solvent, the initiator and the solvent are not particularly limited as long as they are commonly used, and specifically, the initiator may be azobisisobutyronitrile (AIBN) and the solvent may be dimethylformamide (DMF). In addition, a content thereof is not particularly limited as long as it does not impair the physical properties described in the present invention.

In addition, the first polymer may have a number average molecular weight (Mn) of 5,000 to 100,000 Da, specifically, 10,000 to 60,000 Da, and more specifically, 10,000 to 30,000 Da, but is not limited thereto. The number average molecular weight may be controlled by a content ratio and polymerization conditions of the monomer.

Specifically, Chemical Formula 2 according to one embodiment of the present invention may be a copolymer represented by the following Chemical Formula 4 or Chemical Formula 5.

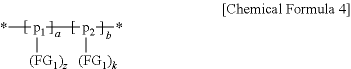

In Chemical Formula 4, FG₁ is an alkynyl functional group, p₁ and p₂ are repeating units derived from a monomer having an FG₁ functional group at the end, z and k are independently an integer of 1 to 7, and a and b are integers of 1 or more. Specifically, z and k may be independently an integer of 1 to 3, and a and b may satisfy 0.1 to 10:1, specifically, 0.5 to 5:1, and more specifically, 0.8 to 2:1, but the present invention is not particularly limited thereto. In addition, the alkynyl functional group may form a triazole ring with the azide functional group of Chemical Formula 3 by a click reaction.

In addition, p₁ and p₂ may be repeating units derived from a monomer having an FG₁ functional group at the end, and specifically, and the monomer is not largely limited as long as it is capable of condensation polymerization or addition polymerization, and specifically, may be one or more monomers selected from acrylic-based, methacrylic-based, and vinyl-based monomers capable of radical polymerization.

In Chemical Formula 4, a and b may refer to the number of moles of each of the p₁ and p₂ repeating units in the first copolymer. A ratio (a:b) of a to b may be controlled by controlling a mole ratio of introduced monomers corresponding to the p₁ and p₂ repeating units or controlling polymerization conditions, but the present invention is not limited thereto.

Specifically, Chemical Formula 4 according to one embodiment of the present invention may be represented by the following Chemical Formula 6.

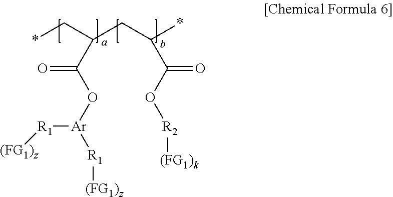

In Chemical Formula 6, Ar is a trivalent aromatic radical, R₁ and R₂ are independently C₁₋₅₀ alkylene, C₃₋₅₀ cycloalkylene, C₆₋₅₀ arylene, C₃₋₅₀ heteroarylene, C₁₋₅₀ alkoxycarbonylene, or a combination thereof, the alkylene, cycloalkylene, arylene, heteroarylene, and alkoxycarbonylene may be optionally substituted with one or more selected from hydroxy, halogen, nitro, cyano, amino, carboxyl, carboxylate, C₁₋₂₀ alkyl, C₂₋₂₀ alkenyl, C₂₋₂₀ alkynyl, C₁₋₂₀ haloalkyl, C₁₋₂₀ alkoxy, C₁₋₂₀ alkoxycarbonyl, C₃₋₃₀ cycloalkyl, (C₆₋₃₀)ar(C₁₋₂₀)alkyl, C₆₋₃₀ aryl, and C₃₋₃₀ heteroaryl, z and k are integers of 1 to 7, and a and b are integers of 1 or more.

Specifically, in Chemical Formula 6, R₁ and R₂ may be independently C₁₋₂₀ alkylene, C₆₋₂₀ arylene, C₁₋₂₀ alkoxycarbonylene, or a combination thereof, the alkylene, arylene, and heteroarylene may be optionally substituted with one or more selected from hydroxy, halogen, carboxyl, C₁₋₇ alkyl, C₁₋₇ haloalkyl, C₁₋₇ alkoxy, C₁₋₇ alkoxycarbonyl, (C₆₋₂₀)ar(C₁₋₇)alkyl, and C₆₋₂₀ aryl, z and k are integers of 1 to 3, and a and b may satisfy 0.1 to 10:1, and specifically, 0.5 to 5:1.

Specifically, Chemical Formula 6 according to one embodiment of the present invention may be represented by the following Chemical Formula 7.

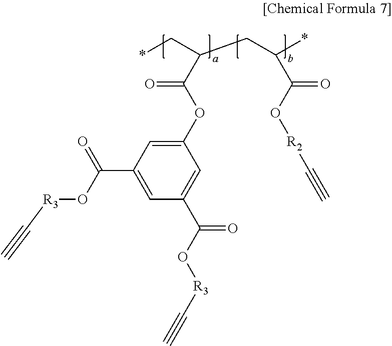

In Chemical Formula 7, R₂ and R₃ are independently a direct bond or C₁₋₁₀ alkylene, and a and b are integers of 1 or more. Specifically, R₂ and R₃ may be independently C₁₋₃ alkylene, more specifically, R₂ and R₃ may be methylene, and a and b may specifically satisfy 0.8 to 2:1.

In addition, z and k in Chemical Formula 4 refer to the number of FG₁ included in each of the p₁ and p₂ repeating units, and taking Chemical Formula 7 as an example, z may be 2 and k may be 1.

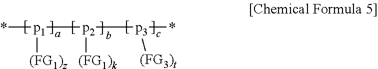

In Chemical Formula 5, FG₁ is an alkynyl functional group, FG₃ is an epoxy functional group, p₁ and p₂ are repeating units derived from a monomer having an FG₁ functional group at the end, p₃ is a repeating unit derived from a monomer having an FG₃ functional group at the end, z, k, and t are independently integers of 1 to 7, and a, b, and c are integers of 1 or more. Specifically, z, k, and t may be independently integers of 1 to 3, and a ratio of the sum of a and b to c (a+b:c) may satisfy a ratio of 1 to 10:1, and preferably, 1 to 7:1, but is not limited thereto. In addition, the epoxy functional group may chemically react with the substrate, and the alkynyl functional group may form a triazole ring through a click reaction with the azide functional group of Chemical Formula 3.

In addition, p₁ and p₂ are repeating units derived from a monomer having an FG₁ functional group at the end, and p₃ may be a repeating unit derived from a monomer having an FG₃ functional group at the end, and specifically, the monomer is not particularly limited as long as it is a monomer capable of condensation polymerization or addition polymerization, and specifically may be one or more monomers selected from acrylic-based, methacrylic-based, and vinyl-based monomers capable of radical polymerization.

In Chemical Formula 5, a to c may refer to the number of moles of the p₁ to p₃ repeating units in the first polymer. A ratio of a to c may be controlled by controlling a molar ratio of monomers corresponding to the p₁ to p₃ repeating units or by controlling polymerization conditions, but is not limited thereto.

Specifically, Chemical Formula 5 according to one embodiment of the present invention may be represented by the following Chemical Formula 8.

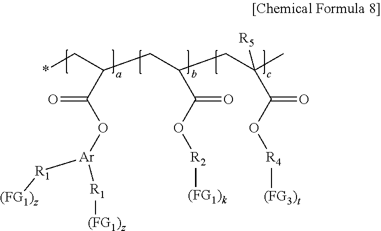

In Chemical Formula 8, Ar is a trivalent aromatic radical, R₁, R₂, and R₄ are independently C₁₋₅₀ alkylene, C₃₋₅₀ cycloalkylene, C₆₋₅₀ arylene, C₃₋₅₀ heteroarylene, C₁₋₅₀ alkoxycarbonylene, or a combination thereof, the alkylene, cycloalkylene, arylene, heteroarylene, and alkoxycarbonylene may be optionally substituted with one or more selected from hydroxy, halogen, nitro, cyano, amino, carboxyl, carboxylate, C₁₋₂₀ alkyl, C₂₋₂₀ alkenyl, C₂₋₂₀ alkynyl, C₁₋₂₀ haloalkyl, C₁₋₂₀ alkoxy, C₁₋₂₀ alkoxycarbonyl, C₃₋₃₀ cycloalkyl, (C₆₋₃₀)ar(C₁₋₂₀)alkyl, C₆₋₃₀ aryl, and C₃₋₃₀ heteroaryl, R₅ is hydrogen or C₁₋₃ alkyl, z, k, and t are integers from 1 to 7, and a, b, and c are integers of 1 or more.

Specifically, in Chemical Formula 8, Ar is a trivalent aromatic radical, R₁, R₂, and R₄ may be independently C₁₋₂₀ alkylene, C₆₋₂₀ arylene, C₁₋₂₀ alkoxycarbonylene, or a combination thereof, the alkylene, arylene, and heteroarylene may be optionally substituted with one or more selected from hydroxy, halogen, carboxyl, C₁₋₇ alkyl, C₁₋₇ haloalkyl, C₁₋₇ alkoxy, C₁₋₇ alkoxycarbonyl, (C₆₋₂₀)ar(C₁₋₇)alkyl, and C₆₋₂₀ aryl, R₅ is hydrogen or methyl, z and k are integers of 1 to 3, and a ratio of the sum of a and b to c (a+b:c) may satisfy a ratio of 1 to 10:1, and preferably, 1 to 7:1.

Specifically, Chemical Formula 8 according to one embodiment of the present invention may be represented by the following Chemical Formula 9.

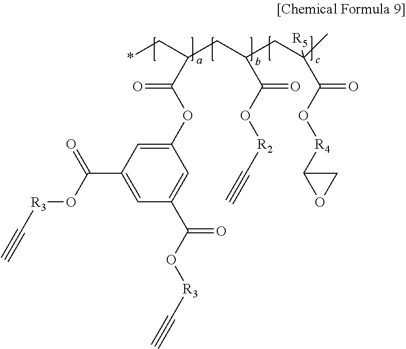

In Chemical Formula 9, R₂ to R₄ are independently C₁₋₁₀ alkylene, R₅ is hydrogen or methyl, and a, b, and c are integers of 1 or more. Specifically, R₂ to R₄ may be independently C₁₋₃ alkylene, R₅ may be methyl, and a ratio of the sum of a and b to c (a+b:c) may satisfy a ratio of 1 to 7:1.

In addition, z, k, and t in Chemical Formula 5 represent the number of FG₁ and FG₃ included in each of the p₁, p₂, and p₃ repeating units, and taking the case of Chemical Formula 9 as an example, z may be 2, k may be 1, and t may be 1.

In Chemical Formulas 4 and 5 according to one embodiment of the present invention, p₁ to p₃ may independently represent repeating units constituting the first polymer of Chemical Formula 2. In Chemical Formulas 4 and 5, the p₁ and p₂ repeating units may be independently derived from a monomer having one or more FG₁ functional groups at the end, and the p₃ repeating unit in Chemical Formula 5 may be derived from a monomer having one or more FG₃ functional groups at the end. The FG₁ functional group is an alkynyl functional group, the FG₃ functional group may be an epoxy functional group, the monomer is not particularly limited in type as long as it is capable of copolymerization, and specifically, the type is not particularly limited as long as it is a monomer capable of condensation polymerization or addition polymerization. Specifically, the monomer may include monomers capable of radical polymerization, such as acrylic-based, methacrylic-based, and vinyl-based monomers, but is not limited thereto.

Specifically, the molar ratio of the monomers introduced for polymerization may be controlled, such that the ratio of a to b of Chemical Formula 4 may be controlled, and the ratio of a to c of Chemical Formula 5 may be controlled. That is, the molar ratio of the corresponding monomers introduced into the polymerization and the ratio of the repeating units p₁ to p₃ may be similar or identical. Specifically, the number of moles of the repeating unit p₁ corresponds to a, the number of moles of p₂ corresponds to b, and the number of moles of p₃ corresponds to c, and when the monomers corresponding to p₁ to p₃ are introduced, respectively, in a molar ratio of 2:2:1 and polymerized, a:b:c may be the same as or similar to 2:2:1, but is not particularly limited thereto, and the ratio may be controlled depending on the reactivity of each monomer and the polymerization conditions.

According to one embodiment of the present invention, the type of the second polymer is not particularly limited as long as it has an azide functional group in a side chain. Specifically, the second polymer may be selected from, but is not limited to, acrylic-based, urethane-based, epoxy-based, fluorene-based, carbazole-based, thiophene-based, and olefin-based polymers. The second polymer may be synthesized by polymerizing one or more monomers, the polymerization may be performed by synthesis in the form of condensation polymerization or addition polymerization, and is not particularly limited, and the monomer may be used without particular limitation as long as it has an azide functional group at the end and may wrap a CNT.

The second polymer may wrap a CNT to produce a second polymer-CNT composite, and specifically, may be a fluorene-based copolymer. Specifically, the fluorene-based copolymer may be obtained by copolymerizing two or more fluorene-based monomers. When the second polymer is a fluorene-based copolymer, which is a conjugated polymer exhibiting electrical conductivity, a CNT may be wrapped more effectively, and therefore, a film formed of high-density CNTs may be manufactured, and a CNT semiconductor device and CNT biosensor having excellent electrical characteristics may be manufactured using the film.

Specifically, the second polymer or the compound represented by Chemical Formula 3 according to one embodiment of the present invention may be a copolymer that simultaneously contains a repeating unit (n) of the following Chemical Formula 10 and a repeating unit (m) of the following Chemical Formula 11.

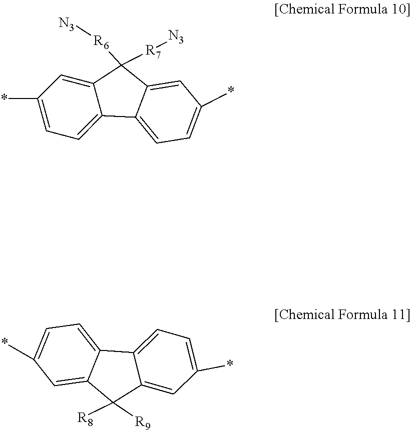

In Chemical Formulas 10 and 11, R₆ and R₇ are independently C₅₋₅₀ alkylene, and R₈ and R₉ are independently C₅₋₅₀ alkyl. Specifically, R₆ and R₇ may be independently C₅₋₂₀ alkylene, R₈ and R₉ may be independently C₅₋₂₀ alkyl, and in the case of alkylene and alkyl satisfying the carbon number in the above range, a CNT may be effectively wrapped through a n-electron interaction (n-n stacking interaction) with a CNT side wall. In particular, when a copolymer containing the repeating unit (n) and the repeating unit (m) is used, a second polymer-CNT composite may be produced by selectively wrapping sc-SWCNT, and a composite layer may be formed using the same, such that a CNT biosensor having further improved electrical performance may be manufactured, which is significantly preferable. In addition, a number average molecular weight of the copolymer containing the repeating unit (n) and the repeating unit (m) may be 1,000 to 500,000 Da, preferably, 3,000 to 50,000 Da, and more preferably, 5,000 to 35,000 Da, but is not limited thereto as long as it does not impair the physical properties targeted in the present invention.

The copolymer containing the repeating unit (n) and the repeating unit (m) may be a random copolymer in which each repeating unit is randomly polymerized, may be an alternating copolymer in which each repeating unit is crossed and bonded, and specifically, may be a random copolymer. When a mole fraction of the repeating unit (n) in the copolymer is n and a mole fraction of the repeating unit (m) is m, n+m may be 1, n may be 0.9 or less or 0.7 or less, preferably, 0.5 or less or 0.4 or less, and more preferably, 0.3 or less, 0.2 or less, or 0.1 or less, and an upper limit thereof is not particularly limited, may be 0.0001 or more, and is not limited thereto as long as it does not impair the physical properties targeted in the present invention. When the above range is satisfied, the copolymer containing the repeating unit (n) and the repeating unit (m) may have further improved selectivity for sc-SWCNTs, such that a CNT film having a higher density of sc-SWCNTs may be coated. In the case of the high-density sc-SWCNT-coated biosensor, it is advantageous because it may exhibit further improved electrical characteristics. The above mole fraction may be used without significant limitation as long as it is a commonly used or known method for analyzing a mole fraction of a copolymer, and may be specifically confirmed through NMR analysis.

According to another aspect of the present invention, the second polymer or the copolymer containing the repeating unit (n) of the second polymer or Chemical Formula 10 and the repeating unit (m) of Chemical Formula 11 may be represented by the following Chemical Formula 14.

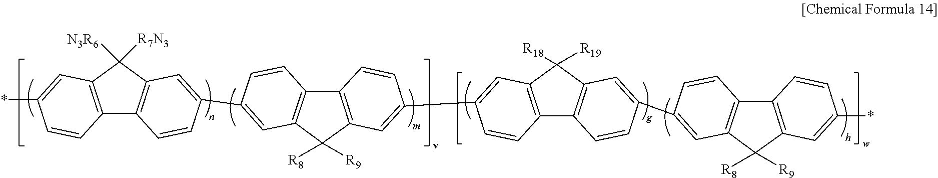

In Chemical Formula 14, R₆ and R₇ are independently C₅₋₂₀ alkylene, R₈, R₉, R₁, and R₁₉ may be independently C₅₋₂₀ alkyl, v, w, n, m, g, and h are independently the mole fractions of the corresponding repeating units in the copolymer, v+w=1, and n+m+g+h=1. Preferably, n may be 0.5 or less or 0.4 or less, and more preferably, 0.3 or less, 0.2 or less, or 0.1 or less, and an upper limit thereof is not significantly limited, and may be 0.0001 or more, but is not limited thereto as long as it does not impair the physical properties targeted in the present invention. In addition, the copolymer may be a random copolymer in which each repeating unit is randomly polymerized, an alternating copolymer in which each repeating unit is crossed and bonded, and specifically, a random copolymer.

A CNT biosensor according to one embodiment of the present invention may further include a self-assembled monolayer (SAM) between the substrate and the polymer layer formed of the first polymer. Specifically, the self-assembled monolayer includes a material which easily reacts with a surface of a substrate layer, as an example, a silane coupling agent, and may be a unit derived from a photopolymerization initiator capable of causing a crosslinking reaction by effectively absorbing energy to form radicals, as an example, a compound having a benzophenone structure.

Specifically, the self-assembled monolayer (SAM) of the CNT biosensor according to one embodiment of the present invention may be a self-assembled monolayer formed of a compound represented by the following Chemical Formula 12.

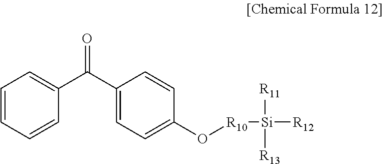

In Chemical Formula 12, R₁₀ is C₁₋₁₀ alkylene, and R₁₁ to R₁₃ are independently hydroxy, halogen, C₁₋₁₀ alkyl, C₁₋₁₀ haloalkyl, C₁₋₁₀ alkoxy, or C₁₋₁₀ alkoxycarbonyl. Specifically, R₁₀ may be C₁₋₇ alkylene, R₁₁ to R₁₃ may be independently halogen, C₁₋₇ alkyl, or C₁₋₇ haloalkyl, the halogen may be Cl or F, and more specifically, Chemical Formula 12 may be represented by the following Chemical Formula 13.

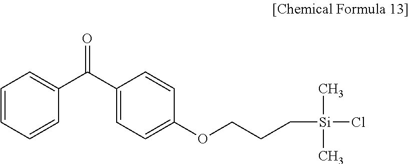 [SMILES: C[Si](C)(Cl)CCCOc1ccc(C(=O)c2ccccc2)cc1]

The compounds represented by Chemical Formulas 12 and 13 have a benzophenone structure to effectively absorb an energy beam, thereby reacting with an alkyl chain of a polymer in contact with electrons in an n-orbital of a carbonyl group of benzophenone. Therefore, the compounds represented by Chemical Formulas 12 and 13 and the first polymer may be cross-linked by energy beam radiation, and as a non-limiting example, the energy beam may be ultraviolet (UV).

The self-assembled monolayer may be formed of the compound represented by Chemical Formula 12, and the self-assembled monolayer is chemically bonded to the substrate and is also cross-linked with the first polymer, thereby fixing the polymer layer formed of the first polymer on the substrate, such that a high-density CNT biosensor that is stable against water and an organic solvent and has excellent reproducibility between devices, which is targeted in the present invention, may be manufactured.

Specifically, the metal electrode of the CNT biosensor according to one embodiment of the present invention may be an electrode formed of one selected from the group consisting of Pt, Al, Au, Cu, Cr, Ni, Ru, Mo, V, Zr, Ti, W, and an alloy thereof, or one selected from the group consisting of indium tin oxide (ITO), Al-doped ZnO (AZO), indium zinc oxide (IZO), F-doped SnO2 (FTO), Ga-doped ZnO (GZO), zinc tin oxide (ZTO), gallium indium oxide (GIO), ZnO, Pd, Ag, and a combination thereof. The metal electrode may include a source electrode and a drain electrode, and a biosensor may be manufactured by forming the metal electrode on the composite layer. A thickness of the metal electrode may be 20 to 100 nm, and preferably 20 to 80 nm, but is not limited thereto.

In one embodiment of the present invention, the antibody layer may be formed on the composite layer, and specifically, the composite layer and the antibody layer may be connected by a linker. In addition, the linker may be connected to the composite layer by a covalent bond or a non-covalent bond.

In one embodiment of the present invention, the linker may be represented by the following Chemical Formula 15.

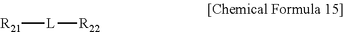

In Chemical Formula 15,
R₂₁ is

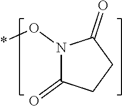

or a polycyclic aromatic hydrocarbon, L is

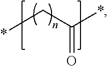

R₂₂ is

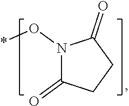

and n is an integer of 1 or more.

Specifically, in Chemical Formula 15, n is an integer of 1 or more, specifically, an integer of 1 to 30, and more specifically, an integer of 1 to 10. In addition, the polycyclic aromatic hydrocarbon may be a C₆₋₆₀ polycyclic aromatic hydrocarbon, and more specifically, may be one or more selected from the compounds shown below, but is not limited thereto as long as it may form a non-covalent bond with the composite layer.

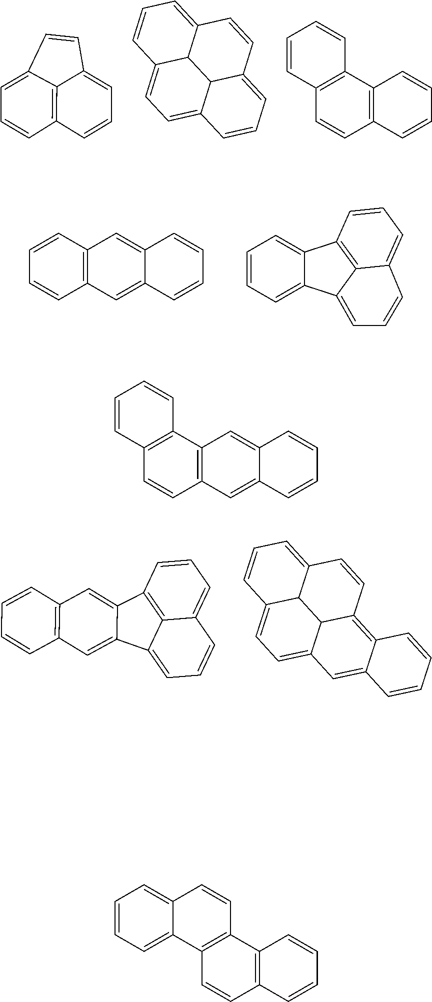

Specifically, the non-covalent bond may be non-covalently bonded through a n-n interaction (stacking interaction) between R₂₁ of the linker and the CNT of the composite layer, or between R₂₁ of the linker and the alkyl chain of the second polymer of the composite layer when R₂₁ of the linker is a polycyclic aromatic hydrocarbon, but is not limited thereto.

In addition, the covalent bond may be formed through a chemical reaction between R₂₁ of the linker and the second polymer of the composite layer, and specifically, the azide functional group at the end of the second polymer of the composite layer may be surface-treated to impart a functional group, and then the functional group and the linker may chemically react to form a covalent bond, more specifically, an amide bond, but is not limited thereto.

According to one embodiment of the present invention, the linker may react with the antibody layer, specifically, R₂₂ of the linker and the antibody may react and be covalently bonded, and thus, the antibody layer and the composite layer may be finally connected using the linker, but are not limited thereto, and the antibody layer may be formed according to a commonly used or known method.

In one embodiment of the present invention, the antibody layer may include one or more types of antibodies capable of antigen-antibody reaction, and optionally may further include one or more combinations selected from enzymes, antigens, aptamers, lectins, nucleic acids, proteins, lipids, sugars, and hormone receptors. The antibody layer may specifically bind to an antigen and detect the antigen. Specifically, the antibody layer may include an antibody that specifically binds to a target biomarker. The biomarker refers to a single molecule or molecules including various metabolites including proteins, DNA, RNA, and the like derived from nucleic acids, and when a specific disease occurs, a unique biomarker for the disease is expressed and acts as a direct indicator of the disease. The target biomarker may be used without significant limitation as long as it is a commonly used or known biomarker, and may be, for example, one or a combination of two or more selected from a biomarker for predicting metabolic syndrome, a biomarker for predicting severe liver fibrosis, a biomarker for diagnosing cardiovascular disease, a biomarker for diagnosing cancer, a biomarker for diagnosing obesity, and a biomarker for predicting or diagnosing neurodegenerative disease.

In one embodiment of the present invention, the biomarker for predicting or diagnosing neurodegenerative disease may be one or more selected from the group consisting of amyloid (A)-β 40, amyloid (A)-β 42, phosphorylated tau protein (p-tau protein), and total tau protein (t-tau protein).

In one embodiment of the present invention, the CNT biosensor may be used for detecting a target biomarker. The CNT-based biosensor according to one embodiment of the present invention may rapidly diagnose a disease by detecting a target biomarker with ultra-high sensitivity.

The present invention provides a method for detecting a target biomarker using the CNT biosensor described above. According to one embodiment of the present invention, the method may include bringing an analyte suspected of containing a biomarker that specifically reacts with or binds to the antibody layer of the biosensor in contact with the biosensor according to one embodiment of the present invention; and measuring binding of the antibody layer and the biomarker. In order to measure the binding of the antibody layer of the biosensor and the biomarker, the measurement may be performed by conversion into a signal using an electrochemical, thermal, optical, or mechanical method, and preferably, the biomarker may be detected through an electrochemical change (current change, resistance change, or the like). As a non-limiting example, when an analyte containing a target biomarker is brought into contact with a biosensor containing an antibody that specifically binds to the target biomarker, the presence of the target biomarker may be confirmed by confirming that a resistance value of the biosensor increases as the antibody and the biomarker react with each other, but is not limited thereto.

The present invention may provide a method for manufacturing a CNT biosensor, the CNT biosensor including: a polymer layer formed of a first polymer on a substrate; a composite layer formed of a second polymer-CNT composite on the polymer layer; a metal electrode formed on the composite layer; and an antibody layer formed on the composite layer, wherein the second polymer-CNT composite is a composite in which a CNT is wrapped by a second polymer, and the polymer layer and the composite layer are connected via triazole.

Specifically, the method for manufacturing a CNT biosensor according to one embodiment of the present invention may include: (a) coating and fixing a first polymer on a substrate; (b) immersing the substrate coated with the first polymer in a second polymer-CNT composite solution; (c) forming a polymer layer and a composite layer by a click reaction between the first polymer and the second polymer; (d) forming a source electrode and a drain electrode on the composite layer; and (e) forming an antibody layer on the composite layer.

In the method for manufacturing a CNT biosensor according to one embodiment of the present invention, the first polymer may be represented by Chemical Formula 2, the second polymer may be represented by Chemical Formula 3, and specific descriptions of the first polymer and the second polymer are the same as described above.

More specifically, in the method for manufacturing a CNT biosensor according to one embodiment of the present invention, the step (a) may include: (a-1) washing the substrate with a solvent; (a-2) coating a self-assembled monolayer (SAM); (a-3) coating the first polymer; (a-4) performing UV curing; and (a-5) washing a compound unfixed to the substrate with a solvent.

In the method for manufacturing a CNT biosensor according to one embodiment of the present invention, the step (a-1) of washing the substrate with the solvent may be performed to remove impurities on the surface of the substrate, and the solvent may be a commonly used inorganic solvent, organic solvent, or a mixture thereof. As a non-limiting example, the solvent may be one or more selected from the group consisting of water, nitric acid, sulfuric acid, hydrogen peroxide, acetone, IPA, THF, benzene, chloroform, methanol, DMF, and toluene, or a mixture thereof, preferably, a first washing may be performed with a mixture of sulfuric acid and hydrogen peroxide, a second washing may be performed with water, and a third washing may be performed with toluene, and a weight ratio of the sulfuric acid and hydrogen peroxide may be 1 to 9:9 to 1, but is not limited thereto.

In the method for manufacturing a CNT biosensor according to one embodiment of the present invention, the step (a-2) of coating the self-assembled monolayer may be performed by a spin coating, dip coating, gas phase deposition, doctor blade coating, or curtain coating method. Specifically, the coating method may be a dip coating method, and the dip coating method may include a process of dipping the washed substrate in a self-assembled monolayer solution for 1 to 20 hours, and preferably, dipping for 3 to 10 hours.

In addition, after completing the dipping process, the washing may be performed with one or two or more solvents selected from the group consisting of acetone, methanol, ethanol, isopropyl alcohol (IPA), toluene, and tetrahydrofuran (THF), and preferably, a first washing with ethanol may be performed, and a second washing with toluene may be performed. The coating of the self-assembled monolayer may be confirmed by measuring a contact angle, and in a case where the contact angle is 40° or more, it may be determined that the self-assembled monolayer is coated.

In the method for manufacturing a CNT biosensor according to one embodiment of the present invention, the self-assembled monolayer solution may contain a compound represented by Chemical Formula 12 and a solvent, and a concentration of the compound represented by Chemical Formula 12 in the self-assembled monolayer solution may be preferably 0.001 to 3 M, but is not particularly limited. In addition, Chemical Formula 12 may be represented by Chemical Formula 13, and the descriptions of Chemical Formulas 12 and 13 are the same as described above.

Specifically, the solvent of the self-assembled monolayer solution may be a solvent that does not react with the compound represented by Chemical Formula 12, and as a non-limiting example, the solvent may be one or more selected from aromatic hydrocarbons including toluene, xylene, and mesitylene; cycloalkanes including cyclohexane, cycloheptane, cyclooctane, and cyclononane; alkanes including hexane, heptane, octane, nonane, and decane; and alkyl alcohols including methanol, ethanol, 1-propanol, and 2-propanol, and is preferably toluene, but is not particularly limited as long as it is a solvent that does not react with the compound represented by Chemical Formula 12.

In the method for manufacturing a CNT biosensor according to one embodiment of the present invention, the step (a-3) of coating the first polymer may be performed by coating the first polymer by one method selected from spin coating, dip coating, dropping, spray coating, solution casting, bar coating, roll coating, and gravure coating, and preferably, the coating method may be selected from spin coating, spray coating, solution casting, and roll coating. In addition, the coating may be performed by preparing a coating solution containing the first polymer. The coating solution may contain the first polymer and a solvent, the solvent is not particularly limited as long as it dissolves the first polymer, and non-limiting examples thereof include one or more selected from ethyl acetate (EA), toluene, acetone, 1,4-dioxane, dimethylacetamide (DMA, N,N-dimethylacetamide), dimethylformamide (DMF), tetrahydrofuran (THF), and chloroform, and preferably 1,4-dioxane or chloroform. The coating solution may contain the first polymer at a concentration of 0.1 to 40 mg/ml, but is not limited thereto, and the concentration may be controlled depending on the desired coating thickness. A coating solution containing the first polymer may be prepared, and an appropriate method may be selected from among the above methods depending on the properties of the coating solution and the intended use.

In the step (a-3) of the method for manufacturing a CNT biosensor according to one embodiment of the present invention, the first polymer may be represented by Chemical Formula 2, and specifically, the first polymer or Chemical Formula 2 may be represented by Chemical Formula 4. Specifically, the first polymer or Chemical Formula 4 may be represented by Chemical Formula 6, and the first polymer or Chemical Formula 6 may be represented by Chemical Formula 7. The descriptions of Chemical Formulas 2, 4, 6, and 7 are the same as described above.

In the method for manufacturing a CNT biosensor according to one embodiment of the present invention, the step (a-4) of performing the UV curing may be performed to fix the polymer layer formed of the first polymer on the substrate by crosslinking the compound represented by Chemical Formula 12 and the first polymer, thereby uniformly coating a CNT film at a high density on a substrate layer; thus, a CNT biosensor that is stable against water and an organic solvent and has excellent reproducibility between devices may be manufactured. The UV curing time may be 0.1 to 30 minutes, but is not limited thereto. For example, the UV curing may be performed using a 365 nm UV lamp, and an intensity of the UV lamp may be 500 to 1,500 mJ/cm², but is not limited thereto.

In addition, the UV curing step may include a process of forming a pattern on a substrate layer through a washing process after forming a UV curing mask pattern using a mask having a pattern formed thereon. The process of forming a pattern has an advantage of being able to form a CNT pattern on the substrate and enabling various circuit designs.

In the method for manufacturing a CNT biosensor according to one embodiment of the present invention, after the UV curing, the substrate layer may be washed with a solvent to remove unreacted compounds. The solvent may be a commonly used solvent, and is not particularly limited as long as it is a solvent that dissolves the unreacted compounds, and as a non-limiting example, one or more solvents selected from toluene, acetone, 1,4-dioxane, EA, DMA, DMF, THF, and chloroform may be used.

In addition, in the method for manufacturing a CNT biosensor according to one embodiment of the present invention, the step (a) may include: (a′-1) washing the substrate with a solvent; (a′-2) coating the first polymer; (a′-3) performing a heat treatment; and (a′-4) washing a compound unfixed to the substrate with a solvent.

In the method for manufacturing a CNT biosensor according to one embodiment of the present invention, the step (a′-1) of washing the substrate with the solvent is performed to remove unreacted organic and inorganic substances remaining on the substrate, and the solvent may be a commonly used inorganic solvent, organic solvent, or a mixture thereof. A specific example of the compound may be the same as or different from the solvent used in the step (a-1) of washing the substrate with the solvent.

The step (a′-2) of coating the first polymer in the method for manufacturing a CNT biosensor according to one embodiment of the present invention may be performed in the same manner as the step (a-3) of coating the first polymer.

In the step (a′-2) of the method for manufacturing a CNT biosensor according to one embodiment of the present invention, the first polymer may be represented by Chemical Formula 2, and specifically, the first polymer or Chemical Formula 2 may be represented by Chemical Formula 5. Specifically, the first polymer or Chemical Formula 5 may be represented by Chemical Formula 8, and more specifically, the first polymer or Chemical Formula 8 may be represented by Chemical Formula 9. The descriptions of Chemical Formulas 2, 5, 8, and 9 are the same as described above.

In the method for manufacturing a CNT biosensor according to one embodiment of the present invention, in the step (a′-3) of performing the heat treatment, the substrate and the first polymer may be chemically bonded to form a polymer layer formed of the first polymer on the substrate. Preferably, a heat treatment temperature may be 100 to 150° C., a heat treatment time may be 1 hour or longer, the temperature and time may be controlled depending on the thickness of the polymer layer and the like, and the ranges of the temperature and time are not particularly limited as long as the physical properties targeted in the present invention are not impaired.

After the heat treatment step, the substrate layer may be washed with a solvent to remove unreacted compounds. The solvent may be a commonly used solvent, and is not particularly limited as long as it is a solvent that dissolves the unreacted compounds, and as a non-limiting example, one or more solvents selected from toluene, acetone, 1,4-dioxane, EA, DMA, DMF, THF, and chloroform may be used.

The second polymer-CNT composite solution according to one embodiment of the present invention may contain a second polymer, a CNT, and a solvent. Specifically, the second polymer-CNT composite solution may be a solution in which a second polymer wrapping a CNT is dissolved in a solvent.

The CNT may be one or more selected from the group consisting of a single-walled carbon nanotube (SWCNT), a double-walled carbon nanotube, a multi-walled carbon nanotube, and a rope carbon nanotube, or may be a single-walled carbon nanotube (SWCNT). Preferably, the CNT may be a conductive single-walled carbon nanotube (m-SWCNT), a semiconducting single-walled carbon nanotube (sc-SWCNT), or a mixture thereof, and when a semiconducting single-walled carbon nanotube (sc-SWCNT) is used, a semiconductor device and a CNT biosensor having more excellent electrical performance may be manufactured, which may be preferable. Depending on the application, a CNT having appropriate physical properties may be selected to form a CNT film on a base substrate. In addition, the CNT may have an outer diameter of 0.1 nm or more, preferably, 0.1 to 10 nm, and more preferably, 0.1 to 5 nm, but is not particularly limited as long as it does not affect the dispersibility during the preparation of the second polymer-CNT composite solution.

The solvent of the second polymer-CNT composite solution is not particularly limited as long as it may dissolve the second polymer of the present invention, and preferably, a non-polar solvent may be used. Non-limiting examples of the non-polar solvent include aromatic hydrocarbon-based solvents such as benzene, toluene, and xylene, and aliphatic hydrocarbon-based solvents such as hexane, heptane, octane, cyclohexane, and methylcyclohexane (MCH), and specifically, toluene or methylcyclohexane may be used. A polar solvent such as chloroform or tetrahydrofuran (THF) may also be used, but is not limited thereto.

A method for preparing a second polymer-CNT composite solution according to one embodiment of the present invention may include a process of dissolving a second polymer in a solvent and then dispersing CNTs. Preferably, the second polymer may be present at a concentration of 0.1 to 30 mg/ml, more preferably, 0.1 to 20 mg/ml, with respect to the solvent, but is not limited thereto, and the CNTs may be present at a concentration of 0.05 to 5 mg/ml. It is preferable that the second polymer is completely dissolved in the solvent, and may be dissolved at a temperature range of 50 to 100° C. Thereafter, the second polymer-CNT composite solution may be prepared by separating the second polymer (second polymer-CNT composite) wrapping carbon nanotubes through centrifugation and performing a filtration process and a redispersion process, but is not limited thereto. In the redispersion process, a concentration of the second polymer-CNT composite in the second polymer-CNT composite solution after the redispersion process may be 0.001 to 10 mg/ml, and a density of the CNT film may be controlled by controlling the concentration, but the present invention is not limited thereto. The solvent used in the redispersion process may be the same as or different from the specific example of the compound of the solvent of the second polymer-CNT composite solution described above, and the dispersion and redispersion processes may be performed through an ultrasonic treatment, but the present invention is not limited thereto. When a substrate is manufactured using a second polymer-CNT composite solution satisfying the above range, a CNT film having an appropriate density may be formed on the substrate layer, and a CNT film having high stability and high density, which is targeted in the present invention, may be manufactured, which is further advantageous, and the prepared second polymer-CNT composite solution may be used in the step (b) of immersing the coated substrate in the second polymer-CNT composite solution.

Specifically, in the step (b), the second polymer may be a copolymer simultaneously containing the repeating unit (n) of Chemical Formula 10 and the repeating unit (m) of Chemical Formula 11, and the description of the copolymer is the same as described above.

In the method for manufacturing a CNT biosensor according to one embodiment of the present invention, the step (c) of forming the polymer layer and the composite layer by the click reaction between the first polymer and the second polymer may be performed by a heating or ultrasonic treatment process in the presence of a copper catalyst. Through the step (c), a polymer layer formed of the first polymer and a composite layer formed of the second polymer-CNT composite may be formed, and the polymer layer and the composite layer may be connected via a triazole ring. Specifically, the step (c) may be performed by an ultrasonic treatment at a temperature of 50 to 60° C. and an intensity of 90 to 120 W in a nitrogen atmosphere, and the ultrasonic treatment may be performed for 1 minute or longer, preferably, 2 minutes to 6 hours, and more preferably, 5 minutes to 2 hours; however, the temperature, intensity, and time are not particularly limited as long as they do not impair the physical properties targeted in the present invention. In addition, even when it is not within the above range, the reaction may be performed by controlling the time in various ways to implement the desired CNT film density. The density of the CNT film may be confirmed by observing the surface of the coating layer using Raman spectroscopy, a scanning electron microscope (SEM), or an optical microscope.

In addition, the process of the click reaction between the first polymer and the second polymer may be represented by Reaction Formula 1, and specifically, may be represented by Reaction Formula 2. The descriptions of Reaction Formulas 1 and 2 are the same as described above.

According to one embodiment of the present invention, when manufacturing a CNT biosensor through the click reaction, a high-density CNT film may be uniformly coated in a short period of time, making it easy and efficient to work, and since the CNT film is coated on the substrate layer through chemical bonding, the adhesion is excellent and stability against water and an organic solvent is secured, preventing the CNT film being peeled off even after washing, which is preferable. In addition, when the click reaction is used, the CNT film is uniformly coated, such that a CNT biosensor having excellent reproducibility between devices and high reliability may be manufactured, which is more preferable.

In the method for manufacturing a CNT biosensor according to one embodiment of the present invention, before the step (d), after completing the reaction, washing unreacted compounds with an organic solvent may be performed, which may be performed to remove unreacted compounds such as catalysts, monomers, and polymers used in the reaction to manufacture a high-purity CNT film. The organic solvent may be used without any special limitation as long as it is a commonly used solvent, and non-limiting examples thereof include one or more solvents selected from toluene, acetone, 1,4-dioxane, EA, DMA, DMF, THF, and chloroform. The washing process may be performed through ultrasonic washing, and the ultrasonic washing may be performed at a strong ultrasonic intensity of 170 to 230 W. The high-density CNT biosensor according to the present invention is significantly advantageous in that it may secure stability against water and an organic solvent by maintaining a high-density CNT film even after an ultrasonic washing process.

In the method for manufacturing a CNT biosensor according to one embodiment of the present invention, the step (d) of forming the source electrode and the drain electrode on the composite layer may be performed using a known or common method for forming an electrode. For example, a method, in which a composite layer formed of the second polymer-CNT composite is subjected to a heat treatment at 100 to 200° C. for 10 to 60 minutes, and then a source electrode and a drain electrode are deposited using a shadow mask, may be used. As a non-limiting example, the source electrode and the drain electrode may be electrodes formed of one or more selected from the group consisting of Pt, Al, Au, Cu, Cr, Ni, Ru, Mo, V, Zr, Ti, W, and an alloy thereof, or one or more selected from the group consisting of indium tin oxide (ITO), Al-doped ZnO (AZO), indium zinc oxide (IZO), F-doped SnO2 (FTO), Ga-doped ZnO (GZO), zinc tin oxide (ZTO), gallium indium oxide (GIO), ZnO, Pd, Ag, and a combination thereof, thicknesses of the source electrode and the drain electrode may be 20 to 100 nm, and preferably, 20 to 80 nm, but are not limited thereto, and the type and thickness of the electrode may be controlled depending on the application.

In the method for manufacturing a CNT biosensor according to one embodiment of the present invention, the step (e) may include (e-1) introducing a linker onto the composite layer; and (e-2) forming an antibody layer by reacting the linker and an antibody. Specifically, in the step (e-1), the composite layer and the linker may be bonded by a non-covalent bond or a covalent bond. The non-covalent bond may be a stacking by a n-n interaction with an electron-rich compound, or a bond by van der Waals attraction, but is not limited thereto, and the details are the same as described above, and thus will be omitted.

In addition, the covalent bond may be induced by surface-treating the azide functional group at the second polymer end of the composite layer to impart a functional group and using the functional group. The surface treatment may be Staudinger reaction, but is not limited thereto. The details are the same as described above, and thus will be omitted.

In the method for manufacturing a CNT biosensor according to one embodiment of the present invention, in the step (e-1), the linker may have a structure represented by Chemical Formula 15.

In one embodiment of the present invention, in the step (e-1), R₂₁ of the linker may be covalently bonded through a chemical reaction with the second polymer of the composite layer or non-covalently bonded through a n-n interaction with the CNT of the composite layer. The specific description is the same as described above, and thus will be omitted.

In one embodiment of the present invention, in the step (e-1), R₂₂ of the linker may be covalently bonded through a reaction with the antibody. The specific description is the same as described above, and thus will be omitted.

In addition, the specific process of introducing the linker in the step (e-1) may be performed according to a commonly used or known method, and specifically, the linker may be dissolved in a solvent at a concentration of 5 to 30 mM, immersed on the composite layer, and reacted for 12 hours or longer, but is not limited thereto.

In addition, the specific process of reacting the antibody in the step (e-2) may be performed according to a commonly used or known method, and for example, may include a process of dropping a solution in which the antibody is dissolved at a concentration of 10 to 500 μg in a buffer solvent (PBS, PBST, or the like) having a pH of 6 to 8 onto the composite layer into which the linker is introduced, performing a reaction at a temperature of 0 to 10° C. for 5 hours or longer, and preferably, 10 to 24 hours, and then performing washing with a solvent and drying. Through this, a biosensor in which an antibody layer is formed may be manufactured. The CNT biosensor according to one embodiment of the present invention has the advantage of being able to exhibit a high CNT density and reproducibility, and furthermore, implementing stable reliability, since the CNT film is stably formed on the substrate, despite the inclusion of the washing process with water, an organic solvent, and the like multiple times.

An acrylate copolymer represented by the following Chemical Formula 6 according to one embodiment of the present invention may be provided.

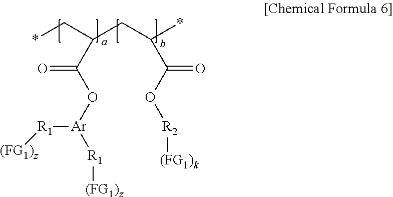

The description of Chemical Formula 6 is the same as described above, and Chemical Formula 6 may be represented by the following compounds, but is not limited thereto.

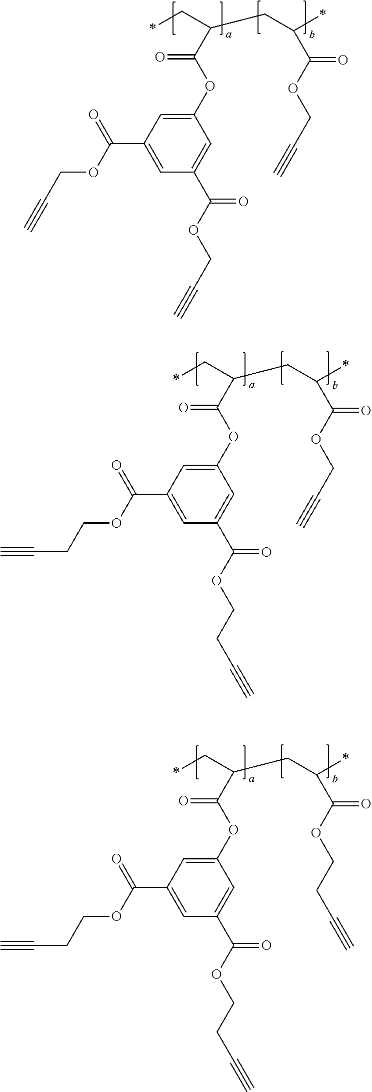

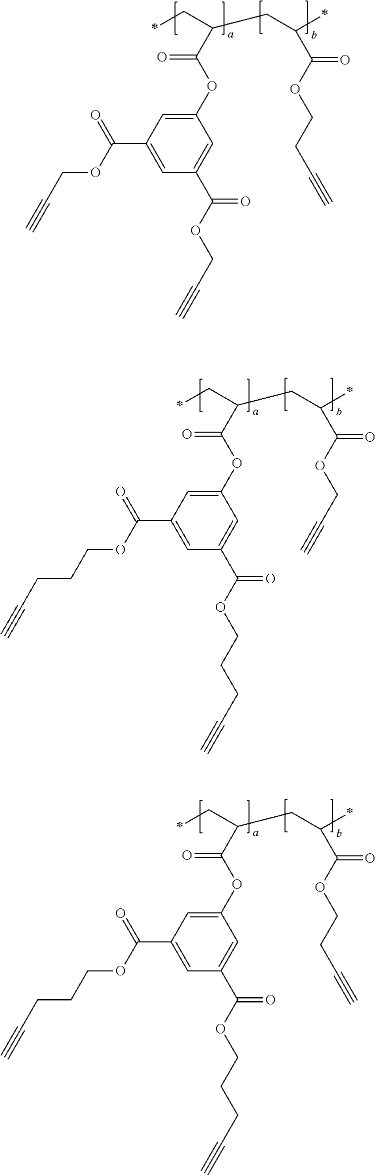

a and b of the compounds are the same as described in Chemical Formula 6.

An acrylate copolymer represented by the following Chemical Formula 8 according to one embodiment of the present invention may be provided.

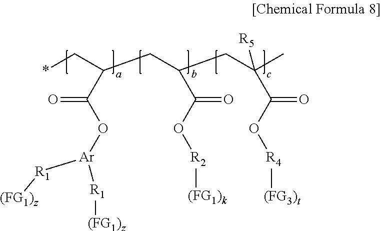

The description of Chemical Formula 8 is the same as described above, and Chemical Formula 8 may be represented by the following compounds, but is not limited thereto.

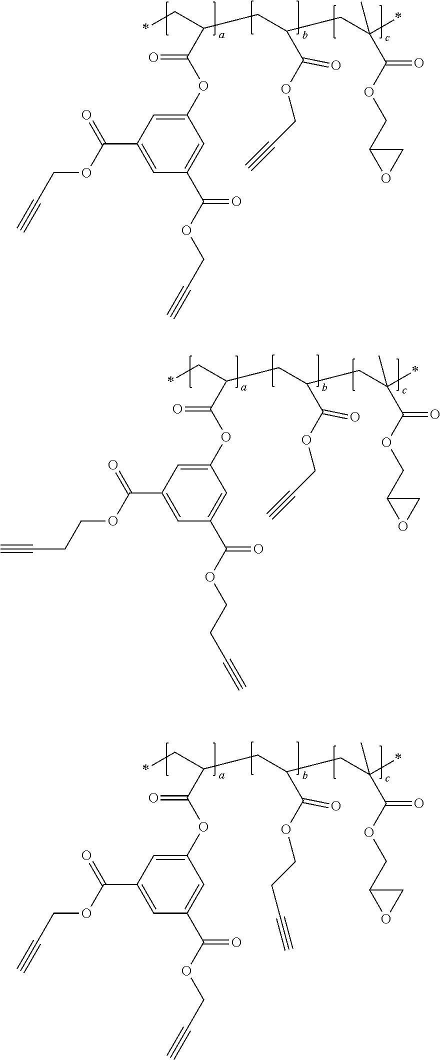

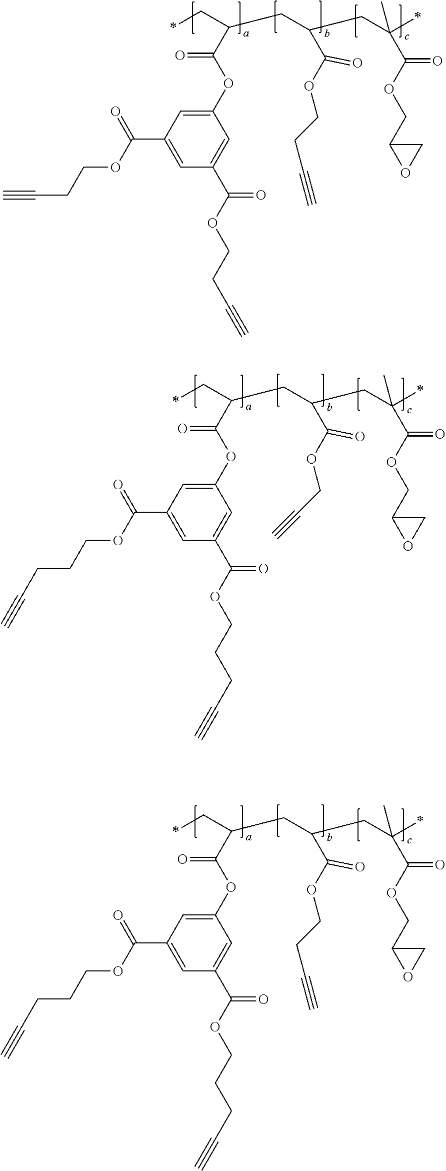

a, b, and c of the compounds are the same as described in Chemical Formula 8.

In addition, in Chemical Formulas 6 and 8 according to one embodiment of the present invention, Ar may be two identical R₁s linked to each other or two different R₁s linked to each other. R₁ may include z FG₁s, and when two different R₁s are linked to Ar, z of the different R₁s may be independently an integer of 1 to 7.

Hereinafter, a high-density CNT biosensor using a click reaction according to the present invention and a method for manufacturing the same will be described in detail by examples. However, the following examples are only a reference to explain the present invention in detail, and the present invention is not limited thereto and may be implemented in various forms. In addition, unless otherwise defined, all the technical terms and scientific terms used have the same meanings as commonly understood by those skilled in the art to which the present invention pertains. In addition, the terms used in the description of the present invention are merely used to effectively describe a specific example, but are not intended to limit the present invention.

In addition, in the following examples and comparative examples, materials whose manufacturers were not listed were purchased and used from Sigma-Aldrich.

### [Preparation Example 1] Preparation of dipropargyl-5-acryloyloxyisophthalate (DPAP) compound

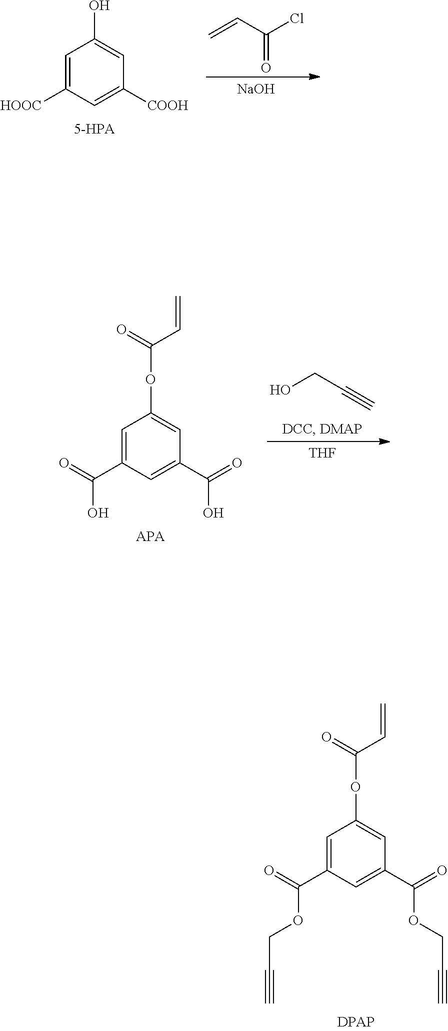

### Preparation of 5-acryloyloxyisophthalic acid (APA)

5.5 mmol (1 g) of 5-hydroxyisophthalic acid (5-HPA) was added to a three-necked flask filled with 10 mL of a 2 M sodium hydroxide (NaOH) solution, and nitrogen purging was performed for 10 minutes. The mixture was cooled to and maintained at 0 to 5° C., 5.8 mmol of acryloyl chloride was added dropwise very slowly for 1 hour, and then stirring was performed at room temperature for 1 hour. HCl was added thereto to precipitate a product of 5-acryloyloxyisophthalic acid (APA). The precipitated product was filtered, washed, and recrystallized with alcohol, and then dried in vacuo at 50° C. for 24 hours to obtain APA (yield: 55%).

¹H NMR spectrum of APA

¹H NMR (300 MHz, DMSO-d₆) δ (ppm): 13.54 (s, 2H), 8.37 (t, J=1.5 Hz, 1H), 7.94 (d, J=1.5 Hz, 2H), 6.59 (dd, J=17.2, 1.5 Hz, 1H), 6.43 (dd, J=17.2, 10.2 Hz, 1H), 6.19 (dd, J=10.2, 1.5 Hz, 1H)

### Preparation of dipropargyl-5-acryloyloxyisophthalate (DPAP)

40 mL of tetrahydrofuran (THF) and 16.34 mmol (3.86 g) of APA were prepared in a flask. 163.4 mmol (8.2 g) of propargyl alcohol and 15 mol % of 4-dimethylaminopyridine (DMAP) with respect to 100 mol of APA were added thereto. The mixture was cooled to 0 to 5° C., and stirred for 1 hour in a nitrogen atmosphere. A solution in which 24.51 mmol (5 g) of N,N′-dicyclohexylcarbodiimide (DCC) was dissolved in 30 mL of THF was added dropwise very slowly thereto, the temperature was slowly raised to room temperature, stirring was further performed for 20 hours, and a precipitate was filtered. Next, the precipitate was dissolved in chloroform and filtered again to remove residual urea. Washing with a 10% aqueous bicarbonate solution was performed three times and purification was performed to obtain a target compound DPAP as white powder (yield: 29%).

¹H NMR, ¹³C NMR, and FT-IR spectra of DPAP

¹H NMR (300 MHz, Chloroform-d) δ (ppm): 8.64 (t, J=1.5 Hz, 1H), 8.05 (d, 2H), 6.66 (m, 1H), 6.34 (m, 1H), 6.09 (m, 1H), 4.96 (d, 4H), 2.55 (t, 2H)

¹³C NMR (75 MHz, Chloroform-d) δ (ppm): 164.13, 164.09, 150.77, 133.95, 131.51, 128.63, 127.92, 127.27, 77.29, 75.68, 53.17

FT-IR (cm⁻¹, KBr): 1731 (ester C═O); 1630 (CH₂═CH—); 3305 (HC≡CH);

### [Preparation Example 2] Preparation of Acrylate Copolymer (i)

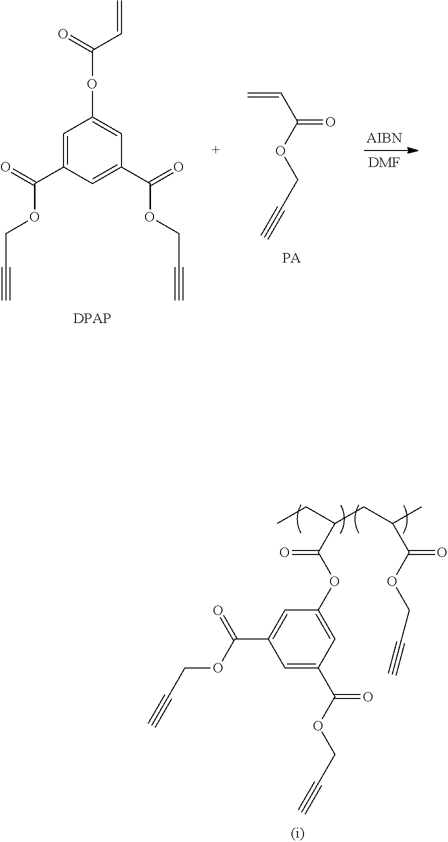

0.8 mmol (0.25 g) of DPAP of Preparation Example 1, 0.8 mmol (0.088 g) of propargyl acrylate (PA), and 0.008 mmol (1.3 mg) of azobisisobutyronitrile (AIBN) were added to a flask with 0.6 mL of dimethylformamide (DMF), and nitrogen purging was performed for 20 minutes. The mixture was stirred at 80° C. for 16 hours to allow polymerization to proceed, a polymerization medium was diluted in dichloromethane, and the reactant was precipitated twice in diethyl ether and dried in vacuo to obtain an acrylate copolymer (i).

The obtained polymer was analyzed with ¹H NMR to confirm that the acrylate copolymer (i) as a target product was prepared, and was analyzed with GPC to confirm that the number average molecular weight (Mn) was 16,762 Da and PDI was 3.3.

¹H NMR (300 MHz, Chloroform-d) δ (ppm): 8.49 (br, 1H), 7.92 (br, 2H), 4.78 (br, 6H), 2.89 (br, 1H), 2.54 (s, 4H)

### [Preparation Example 3] Preparation of Acrylate Copolymer (ii)

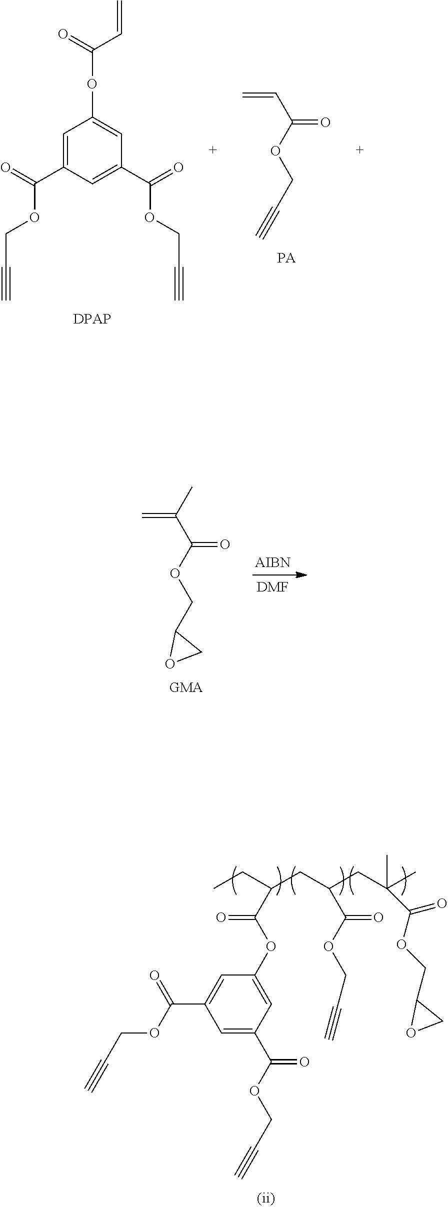

0.8 mmol (0.25 g) of DPAP of Preparation Example 1, 0.8 mmol (0.088 g) of propargyl acrylate (PA), 0.4 mmol (0.057 g) of glycidyl methacrylate (GMA), and 0.001 mmol (1.6 mg) of azobisisobutyronitrile (AIBN) were added to a flask with 0.6 mL of dimethylformamide (DMF), and nitrogen purging was performed for 20 minutes. The mixture was stirred at 80° C. for 16 hours to allow polymerization to proceed. A polymerization medium was diluted in dichloromethane, and the reactant was precipitated twice in diethyl ether and dried in vacuo to obtain an acrylate copolymer (ii).

The obtained polymer was analyzed with ¹H NMR to confirm that the acrylate copolymer (ii) as a target product was prepared, and was analyzed with GPC to confirm that the number average molecular weight (Mn) was 17,800 Da and PDI was 2.11.

¹H NMR (300 MHz, Chloroform-d) δ (ppm): 8.49 (br, 1H), 7.92 (br, 2H), 4.78 (br, 6H), 2.89 (br, 1H), 2.54 (s, 4H)

### [Preparation Example 4] Preparation of Fluorene-Based Copolymer (iii)

### Preparation of 9,9-bis(12-azidododecyl)-2,7-dibromo-9h fluorine monomer

2,7-Dibromo-9H-fluorine (15.43 mmol, 5 g), 1,12-dibromododecane (46 mmol, 15 g), and toluene (60 mL) were added to a shrink flask, nitrogen gas purging was performed, and then, stirring was performed at 80° C. for 20 hours. A reaction mixture was extracted with chloroform, and an organic phase was washed with water and concentrated. A crude product was purified by a column and recrystallized with hexane and ethanol to obtain a compound (X) as a white solid (yield: 63.1%).

¹H NMR (300 MHz, CDCl₃) δ=7.50-7.43 (m, 6H), 3.39 (m, 4H), 1.85 (m, 8H), 1.39 (br, 4H), 1.34-1.10 (m, 18H), 1.00 (s, 10H) 0.61 (br, 4H).

The compound (X) (4.39 mmol, 3.6 g), sodium azide (17.5 mmol, 1.14 g), and dimethylformamide (DMF) (10 mL) were added to a shrink flask, nitrogen gas purging was performed, and stirring was performed at 80° C. for 12 hours. A reaction mixture was extracted with chloroform, and an organic phase was washed with water and concentrated. A crude product was purified by a column and recrystallized with hexane and ethanol to obtain a monomer as a white solid (yield: 82.2%).

¹H NMR (300 MHz, CDCl₃) δ=7.50-7.43 (m, 6H), 3.24 (m, 4H), 1.90 (m, 4H), 1.56 (m, 4H), 1.41-1.0 (br, 32H), 1.09 (br, 10H), 0.56 (br, 4H)

Preparation of Copolymer (iii)

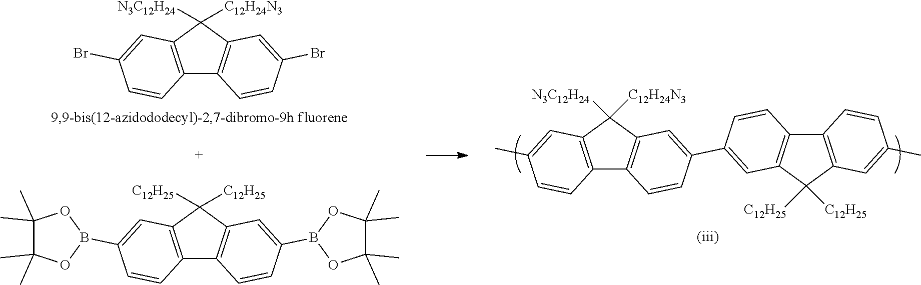

0.5 mmol (0.3774 g) of 2,2′-(9,9-didodecyl-9H-fluorene-2,7-diyl)bis(4,4,5,5-tetramethyl-1,3,2-dioxaborolane) (prepared according to the method of Macromolecules 2018, 51, 3, 755-762), 0.5 mmol (0.3713 g) of the prepared 9,9-bis(12-azidododecyl)-2,7-dibromo-9h fluorene, 0.01 mmol (0.0092 g) of tris(dibenzylideneacetone)dipalladium (Pd₃(dba)₂), 0.04 mmol (0.012 g) of tris(o-tolyl)phosphine, 8 ml of toluene, and 1 ml of tetraethylammonium hydroxide were added to a flask, and nitrogen purging was performed. The mixture was heated to 80° C., and stirring was performed for 20 hours. The resulting mixture was precipitated and filtered using chloroform and methanol to obtain a copolymer (iii) as a yellow solid (yield: 45%). A number average molecular weight of the copolymer was measured to be 28,000 Da.

¹H NMR (300 MHz, CDCl₃) δ=7.85-7.71 (m, 12H), 3.25 (m, 4H), 2.15 (br, 8H), 1.24 (br, 80H), 0.88 (m, 6H)

### [Preparation Examples 5 and 6] Preparation of Fluorene-BASED copolymers (IV and V)

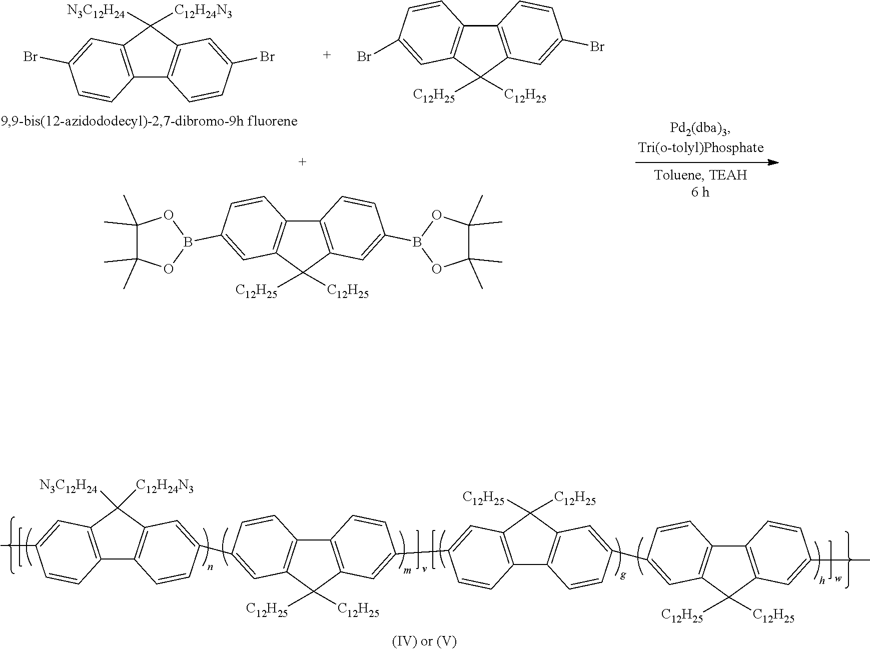

(In the reaction formulas of Preparation Examples 5 and 6, v, w, n, m, g, and h are independently the mole fractions of the corresponding repeating units in the copolymer, v+w=1, and n+m+g+h=1.)

### Preparation of Fluorene-Based Copolymer (IV)

0.25 mmol (0.1887 g) of 2,2′-(9,9-didodecyl-9H-fluorene-2,7-diyl)bis(4,4,5,5-tetramethyl-1,3,2-dioxaborolane) (prepared according to the method of Macromolecules 2018, 51, 3, 755-762), 0.1 mmol (0.0660 g) of 2,7-dibromo-9,9-didodecyl-9H-fluorene (Solarmer), 0.15 mmol (0.1114 g) of the prepared 9,9-bis(12-azidododecyl)-2,7-dibromo-9H-fluorene, 0.005 mmol (0.0048 g) of tris(dibenzylideneacetone)dipalladium (Pd₃(dba)₂), 0.02 mmol (0.0061 g) of tris(o-tolyl)phosphine, 4 ml of toluene, and 0.5 ml of tetraethylammonium hydroxide (TEAH) were added to a flask, and nitrogen purging was performed. The mixture was heated to 85° C., and stirring was performed for 20 hours. The resulting mixture was precipitated and filtered using chloroform and methanol to obtain a copolymer (IV) as a yellow solid (yield: 53%). A number average molecular weight of the copolymer (IV) was measured to be 31,000 Da. It was confirmed through NMR analysis that n was about 0.3.

¹H NMR (300 MHz, CDCl₃) δ=7.83-7.68 (m, 12H), 3.22 (m, 3H), 2.14 (br, 7H), 1.24 (br, 80H), 0.86 (m, 14H)

### Preparation of Fluorene-Based Copolymer (V)

0.25 mmol (0.1887 g) of 2,2′-(9,9-Didodecyl-9H-fluorene-2,7-diyl)bis(4,4,5,5-tetramethyl-1,3,2-dioxaborolane) (prepared according to the method of Macromolecules 2018, 51, 3, 755-762), 0.2 mmol (0.1322 g) of 2,7-dibromo-9,9-didodecyl-9H-fluorene (Solarmer), 0.05 mmol (0.0372 g) of the prepared 9,9-bis(12-azidododecyl)-2,7-dibromo-9H-fluorene, 0.005 mmol (0.0048 g) of tris(dibenzylideneacetone)dipalladium (Pd₃(dba)₂), 0.02 mmol (0.0061 g) of tris(o-tolyl)phosphine, 4 ml of toluene, and 0.5 ml of tetraethylammonium hydroxide (TEAH) were added to a flask, and nitrogen purging was performed. The mixture was heated to 85° C., and stirring was performed for 20 hours. The resulting mixture was precipitated and filtered using chloroform and methanol to obtain a copolymer (V) as a yellow solid (yield: 61%). A number average molecular weight of the copolymer (V) was measured to be 37,000 Da. It was confirmed through NMR analysis that n was about 0.1.

¹H NMR (300 MHz, CDCl₃) δ=7.85-7.68 (m, 12H), 3.22 (m, 1H), 2.13 (br, 9H), 1.24 (br, 80H), 0.86 (m, 18H)

### [Preparation Example 7] Preparation of Second Polymer-CNT Composite Solution

The fluorene-based copolymer (iii) of Preparation Example 4 was added to 20 ml of methylcyclohexane (MCH) at a concentration of 1 mg/ml and heated at 80° C. for 1 hour to completely dissolve. After cooling, 20 mg of purified powder SWCNT (Nanointegris Inc., RN-220) was added and dispersed at room temperature using an ultrasonic processor (Sonics & Materials Inc., VCX-750, 750W), and centrifuged at 85,000 g for 1 hour using a centrifuge (Hanil Scientific Inc., Supra R30). The solution excluding the precipitate was filtered through a 0.20 μm mixed cellulose ester (MCE) membrane to obtain a fluorene-based copolymer (iii) wrapping sc-SWCNT. The obtained pellets were washed several times, added to 10 ml of toluene at a concentration of 0.02 mg/ml, sonicated for 5 minutes, and redispersed to prepare a second polymer-CNT composite solution.

### [Preparation Example 8] Preparation of Self-Assembled Monolayer Solution (BPS Solution)

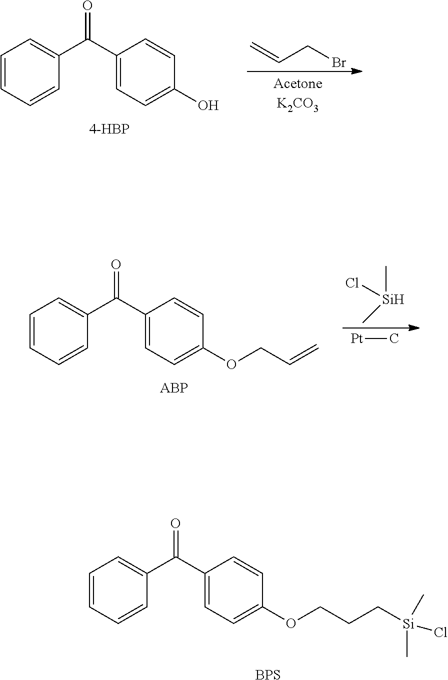

### Preparation of 4-allyloxybenzophenone (ABP)

5.2 mmol (1.02 g) of 4-hydroxybenzophenone (4-HBP) and 7.8 mmol (0.945 g) of allyl bromide were dissolved in 10 mL of anhydrous acetone, and 1.08 g of potassium carbonate (K₂CO₃) was added. The mixture was heated to 75° C., stirred for 8 hours, and then cooled to room temperature. Water was added, the resulting solution was extracted with 50 mL of diethyl ether, washed twice with 50 mL of 10% NaOH, and dried with sodium sulfate (Na₂SO₄), and the solvent was evaporated. Recrystallization was performed with methanol to obtain slightly yellowish ABP (yield: 80%).

¹H NMR spectrum of ABP

¹H NMR (300 MHz, Chloroform-d) δ (ppm): 7.82 (d, J=8.9 Hz, 2H), 7.78-7.72 (m, 2H), 7.60-7.53 (m, 1H), 7.50-7.44 (m, 2H), 6.98 (d, J=8.9 Hz, 2H), 6.14-6.00 (m, 1H), 5.49-5.30 (m, 2H), 4.62 (m, 2H)

### Preparation of 4-(3′-Chlorodimethylsilyl)propyloxybenzophenone (BPS) and BPS solution

2 g of ABP and 20 mL of dimethyl chlorosilane were added to a flask and stirred to prepare a suspension. 10 mg of Pt—C (10% Pt) was added thereto and stirred and refluxed at 50° C. for 8 hours. The reactant was dissolved in toluene at a concentration of 0.01 M, filtered to remove a catalyst, and a BPS solution, which is an oil-type self-assembled monolayer solution containing a BPS compound, was obtained.

¹H NMR spectrum of BPS

¹H NMR (300 MHz, Chloroform-d) δ (ppm): 7.91 (m, 2H), 7.84 (m, 2H), 7.60 (m, 1H), 7.55-7.48 (m, 2H), 7.01 (m, 2H), 4.08-4.00 (m, 2H), 2.02-1.89 (m, 2H), 1.05-0.94 (m, 2H), 0.26-0.21 (s, 6H)

### Example 1

### Manufacturing of CNT Semiconductor Device

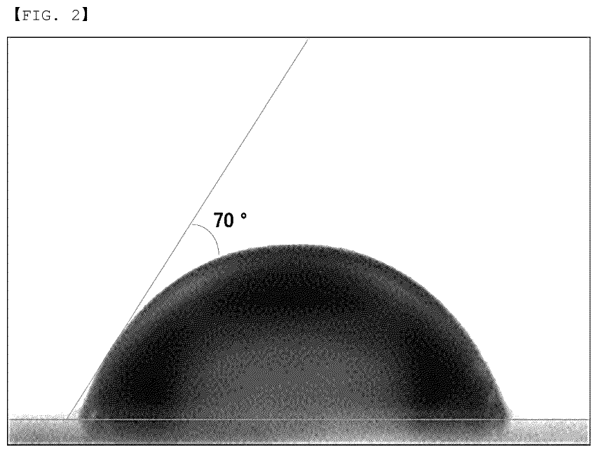

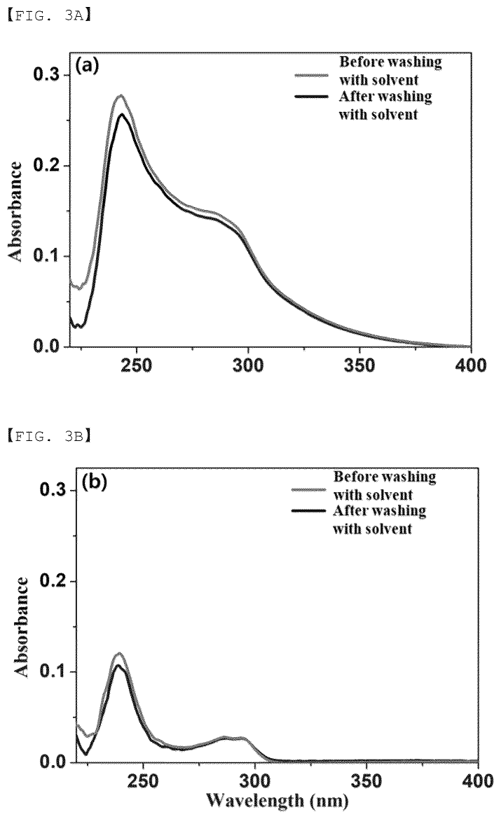

A 100 nm SiO₂ substrate layer (Chung king enterprise) was washed clean with a solution obtained by mixing sulfuric acid (H₂SO₄) and hydrogen peroxide (H₂O₂) at a ratio of 7:3, washed again with water and toluene, and subjected to a heat treatment at 110° C. for 10 minutes in nitrogen gas to completely remove the solvent. The dried substrate layer was immersed in the BPS solution of Preparation Example 8, allowed to be left for 12 hours, and then washed in each of ethanol and toluene for 3 minutes using an ultrasonic cleaner to coat a self-assembled monolayer (SAM). The SAM-coated substrate layer was spin-coated with a solution in which the acrylate copolymer (i) of Preparation Example 2 was dissolved in 1,4-dioxane at a concentration of 5 mg/ml under conditions of 1,000 rpm for 50 seconds, and was UV-cured at an intensity of 727 mJ/cm² for 1 minute to perform a substrate fixation step. Next, the compound unfixed to the substrate layer was removed by ultrasonic washing in chloroform for 1 hour, the solvent was removed with nitrogen gas, and a heat treatment was performed at 100° C. for 10 minutes. At this time, as seen in FIG. 2, the contact angle measurement result for water was confirmed to be 70° or more, thereby confirming the presence or absence of self-assembled monolayer (SAM) coating, and as seen in FIG. 3A, the results before and after coating the acrylate copolymer (i) solution, performing UV curing, and washing the substrate layer with chloroform were compared through ultraviolet-visible spectroscopy (UV-Vis spectroscopy).

The substrate layer coated with the acrylate copolymer (i) was added to a vial and immersed in 1 ml of the second polymer-CNT composite solution of Preparation Example 7, 0.003 g of copper sulfate (CuSO₄), 0.019 g of sodium ascorbate, and 0.5 ml of distilled water were added, and nitrogen purging was performed. The vial was placed in an ultrasonic cleaner and ultrasonicated at a temperature of 50° C. and an intensity of 110 W for 5 minutes to perform a click reaction. After completing the reaction, ultrasonic washing was performed in toluene to remove compounds unreacted with the substrate, the solvent was removed with nitrogen gas, and then, a heat treatment was performed at 150° C. for 30 minutes.

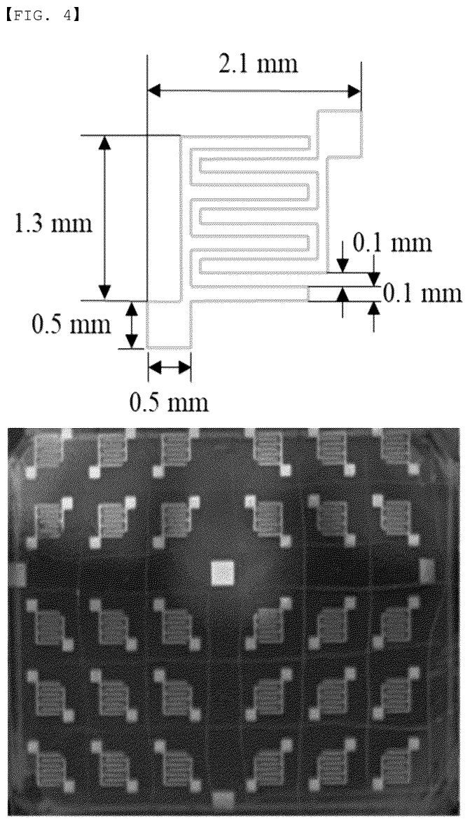

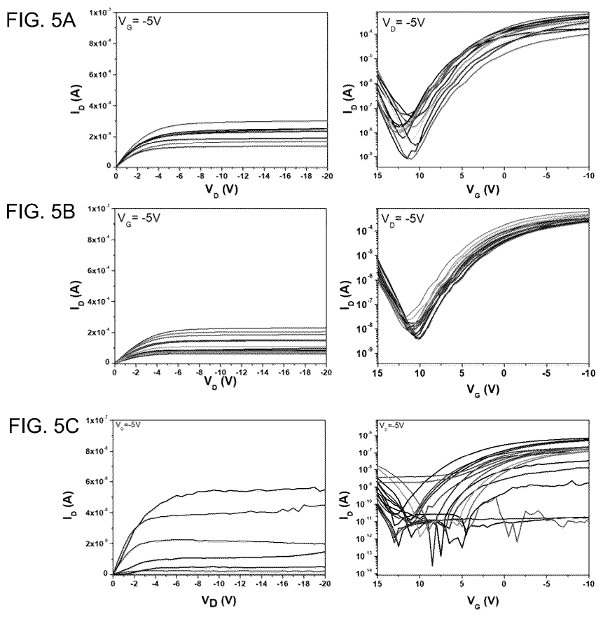

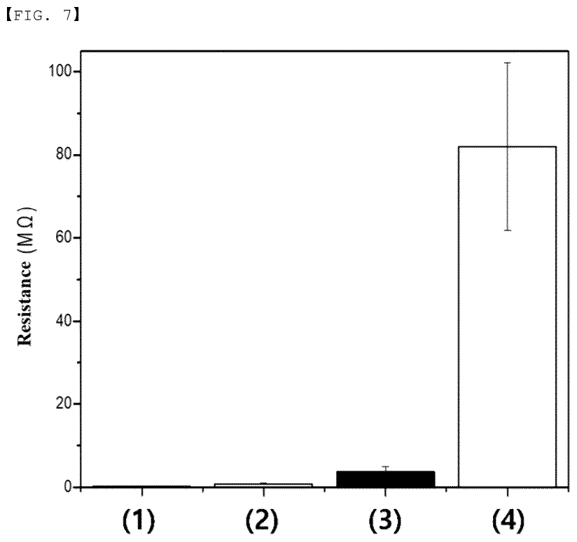

A source electrode and a drain electrode were formed by depositing Au to a thickness of 60 μm on a completely dried substrate using a shadow mask, thereby manufacturing a CNT semiconductor device. The shadow mask used is illustrated in FIG. 4. Electrical characteristic curves (output curves and transfer curves) of the manufactured CNT semiconductor device were measured and are illustrated in FIG. 5A. In addition, changes in current according to the voltage of the CNT semiconductor device were measured, an average resistance value was calculated, and the results are illustrated in (1) of FIG. 7.

### Manufacturing of CNT Biosensor

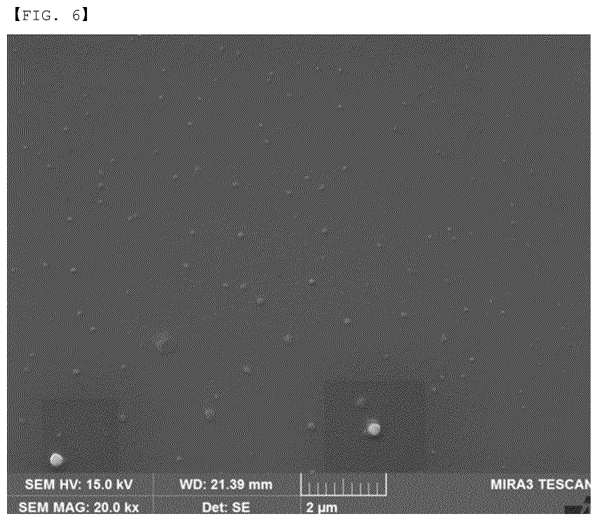

The manufactured CNT semiconductor device was immersed in a 10 mM 1-pyrenebutanoic acid succinimidyl ester (linker) solution for 12 hours or longer and then washed with DMF to manufacture a CNT semiconductor device into which a linker was introduced. Changes in current according to the voltage of the manufactured CNT semiconductor device were measured, an average resistance value was calculated, and the results are illustrated in (2) of FIG. 7. In addition, an SEM image of a surface of the CNT semiconductor device into which a linker was introduced is illustrated in FIG. 6, and as seen in FIG. 6, it was confirmed that the linker was stably formed on the composite layer even after washing.

Subsequently, 100 μg/ml of an Aβ antibody solution dissolved in phosphate-buffered saline (PBS, pH 7.4) was dropped between the electrodes of the semiconductor device and reacted at 4° C. for 12 hours, and then washing was performed with phosphate buffered saline with Tween 20 (PBST) to remove unreacted antibodies. Bovine serum albumin (BSA, 3 wt %) dissolved in PBST was dropped and reacted at 4° C. for 1.5 hours, and then washed with PBST for 10 minutes or longer to remove unreacted BSA, thereby finally manufacturing a CNT biosensor according to Example 1. Changes in current according to the voltage of the CNT biosensor were measured, an average resistance value was calculated, and the results are illustrated in (3) of FIG. 7.

### Example 2

A 100 nm SiO2 substrate layer (Chung king enterprise) was washed clean with a solution obtained by mixing sulfuric acid (H₂SO₄) and hydrogen peroxide (H₂O₂) at a ratio of 7:3, washed again with water and toluene, and subjected to a heat treatment at 110° C. for 10 minutes in nitrogen gas to completely remove the solvent. The dried substrate layer was spin-coated with a solution in which the acrylate copolymer (ii) of Preparation Example 3 was dissolved in 1,4-dioxane at a concentration of 5 mg/ml under conditions of 1,000 rpm for 50 seconds, and was subjected to a heat treatment at 110° C. for 2 hours to perform a substrate fixation step. Next, the compound unfixed to the substrate layer was removed by ultrasonic washing in chloroform for 1 hour, the solvent was removed with nitrogen gas, and a heat treatment was performed at 100° C. for 10 minutes. As illustrated in FIG. 3B, the results before and after coating the acrylate copolymer (ii) solution, performing heat curing, and washing the substrate layer with chloroform were compared through ultraviolet-visible spectroscopy (UV-Vis spectroscopy).

The substrate layer coated with the acrylate copolymer (ii) was added to a vial and immersed in 1 ml of the second polymer-CNT composite solution of Preparation Example 7, 0.003 g of copper sulfate (CuSO4), 0.019 g of sodium ascorbate, and 0.5 ml of distilled water were added, and nitrogen purging was performed. The vial was placed in an ultrasonic cleaner and ultrasonicated at a temperature of 50° C. and an intensity of 110 W for 5 minutes to perform a click reaction. The subsequent steps were performed in the same manner as in Example 1, and finally, a CNT semiconductor device and a CNT biosensor according to Example 2 were manufactured. The shadow mask used was the same as that used in Example 1, and the electrical characteristic curves (output curves and transfer curves) for the CNT semiconductor device manufactured according to Example 2 are illustrated in FIG. 5B.

### Comparative Example 1

In Example 1, the substrate layer coated with the acrylate copolymer (i) was spin-coated with the second polymer-CNT composite solution of Preparation Example 7 under conditions of 2,000 rpm rather than a click reaction, and then dried on a hot plate. The process was repeated twice to coat the film, and then ultrasonically washed in toluene to remove compounds unreacted with the substrate, the solvent was removed with nitrogen gas, and then, a heat treatment was performed at 150° C. for 30 minutes. A CNT semiconductor device according to Comparative Example 1 was manufactured by forming a source electrode and a drain electrode by depositing Au to a thickness of 60 μm on a completely dried substrate using a shadow mask. The electrical characteristic curves (the output curves and the transfer curves) for all the devices of the CNT biosensor according to Comparative Example 1 are illustrated in FIG. 5C.

The electrical characteristic curves (the output curves and the transfer curves) of the semiconductor devices according to Examples 1 and 2 and Comparative Example 1 were measured at room temperature using an I-V measurement software source measure unit (B1500A, Agilent), and the results are illustrated in FIG. 5. As seen in FIG. 5, in the semiconductor devices using the click reaction according to Examples 1 and 2, it was confirmed that the output curves and the transfer curves were measured uniformly, and reproducibility and reliability between the devices were ensured. On the other hand, in Comparative Example 1, it was confirmed that the measurement results between the devices were uneven.

### [Evaluation Examples] Evaluation of Biomarker Detection Performance of Biosensor

A 10 pM Aβ antigen solution was prepared by adding HFIP-treated Aβ42 at a concentration of 4 mM dissolved in DMSO to PBS. The CNT biosensors manufactured according to Examples 1 and 2 and Comparative Example 1 were sufficiently washed with PBST and PBS and dried, and then, the antigen solution was dropped and reacted at 4° C. for 2 hours. The reaction-completed CNT biosensors were sufficiently washed with PBST and PBS and dried again, changes in current according to the voltage were measured, an average resistance value was calculated, and the results are illustrated in (4) of FIG. 7.

The resistance changes calculated through the current change according to the voltage of the sample of Example 1 at each process step showed an average resistance value of 0.001 MΩ in (1), 0.5 MΩ in (2), 4 MΩ in (3), and 80 MΩ in (4), as seen in FIG. 7, whereas the results of the sample of Comparative Example 1 were irregular each time the measurement was performed. Through this, it was confirmed that the CNT biosensor according to one embodiment of the present invention has excellent biomarker detection performance even at a low biomarker concentration.

The CNT biosensor according to the present invention is stable against water and an organic solvent because the CNT is hardly peeled off even when the washing process is performed several times, and at the same time, reproducibility between the devices may be easily secured. The biosensor has the advantage of being able to implement excellent reliability since it is manufactured using a semiconductor device in which electrical characteristic curves between the devices are uniform.

In the present invention, in order to solve the problems of conventional CNT-based biosensors that have the problem of easy peeling, are vulnerable to an organic solvent, and have low reliability between devices, a CNT biosensor was manufactured using a click reaction, and the semiconductor device having excellent reproducibility between devices and high reliability may be widely utilized in the field of diagnostic devices such as biosensors.

Hereinabove, although the present invention has been described by specific matters and limited examples and comparative examples, they have been provided only for assisting in the entire understanding of the present invention. Therefore, the present invention is not limited to the examples, and various modifications and changes may be made by those skilled in the art to which the present invention pertains from this description.

Therefore, the spirit of the present invention should not be limited to the described examples, but the claims and all modifications equal or equivalent to the claims are intended to fall within the spirit of the present invention.

1. A CNT biosensor comprising:
a polymer layer formed of a first polymer on a substrate;
a composite layer formed of a second polymer-CNT composite on the polymer layer;
a metal electrode formed on the composite layer; and
an antibody layer formed on the composite layer,
wherein the second polymer-CNT composite is a composite in which a CNT is wrapped by a second polymer, and
the polymer layer and the composite layer are connected via triazole.

2. The CNT biosensor of claim 1, wherein the composite layer and the antibody layer are connected by a linker.

3. (canceled)

4. The CNT biosensor of claim 1, wherein the triazole is represented by the following Chemical Formula 1: 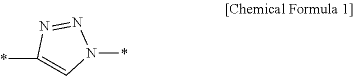 [SMILES: *c1cn(*)nn1]
wherein
the asterisks (*) are each independently a connection point with the first polymer of the polymer layer or the second polymer of the composite layer, and the two asterisks (*) are connection points between different layers.

5. The CNT biosensor of claim 1, wherein the first polymer is represented by the following Chemical Formula 2,
the second polymer is represented by the following Chemical Formula 3,
the triazole is formed by a click reaction between the first polymer and the second polymer, and the click reaction is a reaction represented by the following Reaction Formula 1: 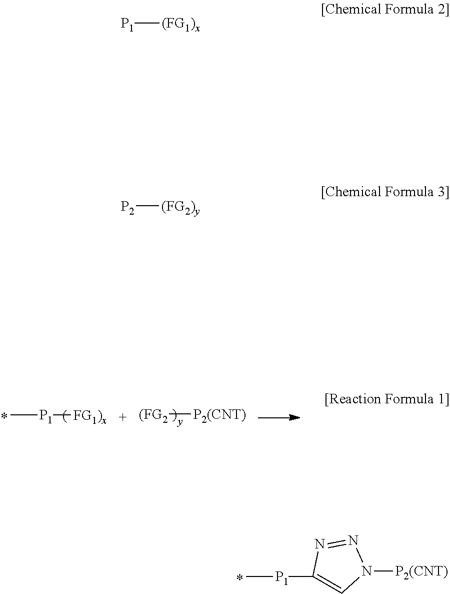
wherein
P₁ is a residue derived from the first polymer;
* is a moiety where P₁ is fixed to the substrate;
P₂ is a residue derived from the second polymer;
P₂(CNT) is a residue derived from the second polymer-CNT composite;
FG₁ is an alkynyl functional group;
FG₂ is an azide functional group; and
x and y are integers of 1 or more.

6. The CNT biosensor of claim 1, wherein the first polymer is an acrylic-based copolymer.

7. The CNT biosensor of claim 5, wherein Chemical Formula 2 is represented by the following Chemical Formula 4 or Chemical Formula 5: 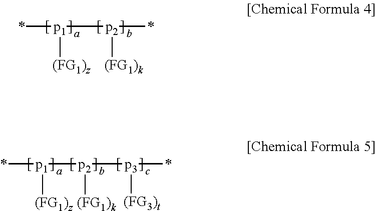
wherein
FG₁ is an alkynyl functional group;
FG₃ is an epoxy functional group;
p₁ and p₂ are repeating units derived from a monomer having an FG₁ functional group at the end;
p₃ is a repeating unit derived from a monomer having an FG₃ functional group at the end;
z, k, and t are integers of 1 to 7; and
a, b, and c are integers of 1 or more.

8. The CNT biosensor of claim 7, wherein Chemical Formula 4 is represented by the following Chemical Formula 6, and
Chemical Formula 5 is represented by the following Chemical Formula 8: 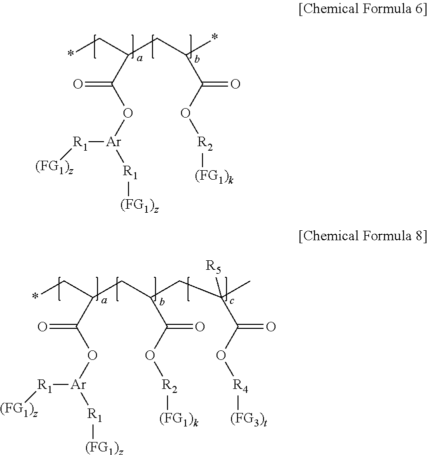
wherein
Ar is a trivalent aromatic radical;
R₁, R₂, and R₄ are independently C₁₋₅₀ alkylene, C₃₋₅₀ cycloalkylene, C₆₋₅₀ arylene, C₃₋₅₀ heteroarylene, C₁₋₅₀ alkoxycarbonylene, or a combination thereof,
the alkylene, cycloalkylene, arylene, heteroarylene, and alkoxycarbonylene may be optionally substituted with one or more selected from hydroxy, halogen, nitro, cyano, amino, carboxyl, carboxylate, C₁₋₂₀ alkyl, C₂₋₂₀ alkenyl, C₂₋₂₀ alkynyl, C₁₋₂₀ haloalkyl, C₁₋₂₀ alkoxy, C₁₋₂₀ alkoxycarbonyl, C₃₋₃₀ cycloalkyl, (C₆₋₃₀)ar(C₁₋₂₀)alkyl, C₆₋₃₀ aryl, and C₃₋₃₀ heteroaryl;
FG₁ is an alkynyl functional group;
FG₃ is an epoxy functional group;
z, k, and t are independently an integer of 1 to 7; and
a, b, and c are independently an integer of 1 or more.

9. The CNT biosensor of claim 8, wherein Chemical Formula 6 is represented by the following Chemical Formula 7, and
Chemical Formula 8 is represented by the following Chemical Formula 9: 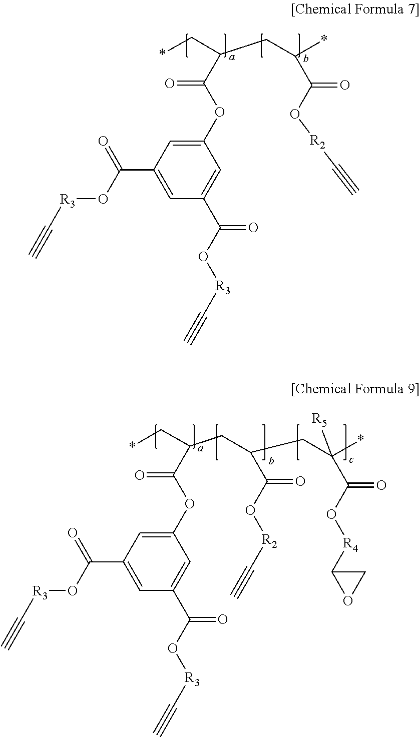
wherein
R₂ to R₄ are independently C₁₋₁₀ alkylene;
R₅ is hydrogen or methyl; and
a, b, and c are independently an integer of 1 or more.

10. (canceled)

11. The CNT biosensor of claim 5, wherein Chemical Formula 3 is a copolymer containing a repeating unit (n) of the following Chemical Formula 10 and a repeating unit (m) of the following Chemical Formula 11: 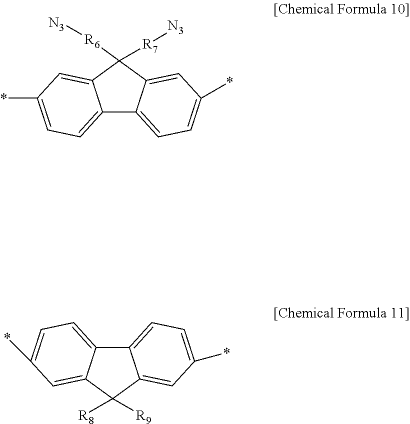
wherein
R₆ and R₇ are independently C₅₋₅₀ alkylene; and
R₅ and R₉ are independently C₅₋₅₀ alkyl.

12. The CNT biosensor of claim 1, wherein the CNT in the second polymer-CNT composite is a semiconducting single-walled carbon nanotube (sc-SWCNT).

13. The CNT biosensor of claim 1, wherein the antibody layer includes an antibody that specifically binds to a target biomarker.

14. The CNT biosensor of claim 13, wherein the target biomarker is one or a combination of two or more selected from a biomarker for predicting metabolic syndrome, a biomarker for predicting severe liver fibrosis, a biomarker for diagnosing cardiovascular disease, a biomarker for diagnosing cancer, a biomarker for diagnosing obesity, and a biomarker for predicting or diagnosing neurodegenerative disease.

15. (canceled)

16. A method for manufacturing a CNT biosensor, the CNT biosensor including:
a polymer layer formed of a first polymer on a substrate;
a composite layer formed of a second polymer-CNT composite on the polymer layer;
a metal electrode formed on the composite layer; and
an antibody layer formed on the composite layer,
wherein the second polymer-CNT composite is a composite in which a CNT is wrapped by a second polymer, and
the polymer layer and the composite layer are connected via triazole.

17. The method of claim 16, wherein the method includes:
(a) coating and fixing the first polymer on the substrate;
(b) immersing the substrate coated with the first polymer in a second polymer-CNT composite solution;
(c) forming a polymer layer and a composite layer by a click reaction between the first polymer and the second polymer;
(d) forming a source electrode and a drain electrode on the composite layer; and
(e) forming an antibody layer on the composite layer.

18. The method of claim 17, wherein the step (e) includes:
(e-1) introducing a linker onto the composite layer; and
(e-2) forming an antibody layer by reacting the linker and an antibody.

19. The method of claim 18, wherein the linker is represented by the following Chemical Formula 15: 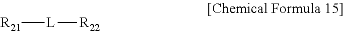
wherein
R₂₁ is 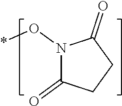
or a polycyclic aromatic hydrocarbon,
L is 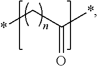
R₂₂ is 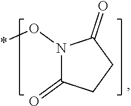
and
n is an integer of 1 or more.

20. (canceled)

21. The method of claim 19, wherein in the step (e-1), R₂₂ of the linker is covalently bonded through a reaction with the antibody.

22. The method of claim 17, wherein the step (a) includes:
(a-1) washing the substrate with a solvent;
(a-2) coating a self-assembled monolayer (SAM);
(a-3) coating the first polymer;
(a-4) performing UV curing; and
(a-5) washing a compound unfixed to the substrate with a solvent.

23. (canceled)

24. The method of claim 22, wherein the step (a-4) further includes forming a pattern.

25. The method of claim 17, wherein the step (a) includes:
(a′-1) washing the substrate with a solvent;
(a′-2) coating the first polymer;
(a′-3) performing a heat treatment; and
(a′-4) washing a compound unfixed to the substrate with a solvent.

26. (canceled)
